(19) United States
(12) Patent Application Publication
(10) Pub. No.: US 2026/0110030 A1
(43) Pub. Date: Apr. 23, 2026

(54) NUCLEIC ACID ANALYSIS OF PERFUSION OR FLUSH FLUIDS

(71) Applicant: Natera, Inc., San Carlos, CA (US)

(72) Inventors: Paul VAN HUMMELEN, San Francisco, CA (US); Phil GAUTHIER, Englewood, CO (US)

(73) Assignee: Natera, Inc., San Carlos, CA (US)

(21) Appl. No.: 19/117,583

(22) PCT Filed: Sep. 27, 2023
(86) PCT No.: PCT/US23/33880

§ 371 Date: Apr. 1, 2025

Related U.S. Application Data
(60) Provisional application No. 63/413,834, filed on Oct. 6, 2022.

(51) Int. Cl. C12Q 1/6881

(52) U.S. Cl. CPC C12Q 1/6881; C12Q 2600/118

(57) ABSTRACT

Systems and methods are provided herein for preparing a non-naturally-occurring preparation of DNA from perfusion fluid or flush, the preparation being useful for assessing transplantation outcomes of a donor organ or donor tissue which was perfused with the perfusion fluid or prepared for transplantation with flush. The preparation may also be useful for evaluating quality of the organ or tissue. Analysis of the DNA may be used to make clinical decisions. Analysis of the DNA may also be used for adaptive control of a mechanical perfusion system used for preserving the organ or tissue, by generating and executing appropriate parameter adjustments based on characteristics of the sampled DNA.

### CROSS-REFERENCE TO RELATED APPLICATION

This application claims priority to and the benefit of U.S. Provisional Patent Application No. 63/413,834, filed Oct. 6, 2022, which is incorporated herein by reference in its entirety.

### BACKGROUND

In the field of organ transplantation, it is often necessary to preserve an organ in a controlled manner after it has been removed from a donor and prior to transplantation into a recipient, in order to keep the organ viable and to prevent injury to the organ and graft failure. Common preservation methods include the standard-of-care static cold storage (SCS) and various dynamic perfusion techniques. Dynamic perfusion of organs seeks to mimic the physiological environment of the body by passing fluid through the organ in a circulatory manner.

Due to improved outcomes, mechanical perfusion has been increasingly used instead of SCS, especially for high-risk or marginal organs such as recovered deceased-donor kidneys. SCS involves subjecting the organ to hypoxic conditions which leads to the accumulation of metabolites via anaerobic metabolism. This buildup of metabolites can increase the risk of ischemia-reperfusion injury (IRI) upon reperfusion of the organ in the recipient, so it is desirable to use a preservation method that can regulate the levels of metabolites in the organ, as well as re-supply oxygenated blood/oxygen carrier, medication, and essential nutrients. Furthermore, mechanical perfusion at normothermic temperatures allows for re-conditioning donor organs prior to transplantation, such that blood flow can be restored earlier and improve recipients' outcomes. However, perfused organs can still undergo injury that leads to negative transplantation outcomes, so there exists a need for a method of assessing organ quality or status during perfusion prior to transplantation.

Additionally, all solid organs that are preserved using SCS are flushed with a crystalloid solution in the operating room prior to implantation. The purpose of this flush is to remove blood clots and other organic debris from the donor organ circulatory system prior to anastomosis to recipient circulatory system.

### SUMMARY

The systems, devices, and methods described herein allow for analysis of nucleic acids sampled from perfusate, the fluid used for mechanical perfusion of an organ or tissue, or from organ flush, received from a donor. While both fluids are used to preserve and/or re-condition the organ/tissue in storage or transportation prior to transplantation into a recipient, the perfusate may be sampled, according to the methods described herein, in order to isolate and analyze DNA. Perfusion and flush fluids are typically thrown away, so sampling, manipulation, and analysis of these fluids is neither routine nor conventional. Nucleic acids, for example, cell-free DNA (cfDNA), cellular DNA, or RNA, from the perfusate may be quantified, enriched to generate unnatural preparations for purposes of analysis, sequenced, amplified to generate unnatural preparations for purposes of analysis, or otherwise manipulated to generate unnatural preparations for purposes of analysis in order to inform physicians as to a current status of the perfused organ/tissue. Analytical results derived from such preparations, such as cfDNA concentration or fragment size distribution (or a subset thereof) may indicate that the organ/tissue has been injured and may no longer be suitable for transplantation or may require adjustments to the perfusion. The analytical results arising from such preparations may also be indicative of a quality of the donor organ/tissue that can be used to assess its suitability for transplantation and for informing the process of allocating the organ/tissue to a suitable recipient. The analytical results may be predictive of primary graft dysfunction after transplantation of the organ/tissue, predictive of delayed graft function in kidney transplant recipients, and/or predictive of overall post-transplant organ function.

In a first aspect, provided herein is a method for preparing a nucleic acid preparation from perfusion fluid useful for predicting transplantation outcome of a donor organ or a donor tissue. The method includes obtaining a sample of perfusion fluid from a donor organ or a donor tissue that has been perfused with the perfusion fluid, the perfusion fluid including nucleic acids; isolating nucleic acids from the sample; and performing an analysis of the isolated DNA to evaluate at least one of an amount of nucleic acids in the perfusion fluid, a molecular weight of DNA in the perfusion fluid, or a fragment size distribution of nucleic acids in the perfusion fluid.

In some implementations, the amount of nucleic acids is a total amount of nucleic acids in the perfusion fluid. In some implementations, the amount of nucleic acids is an amount of high molecular-weight DNA (e.g., greater than 200 base pairs) in the perfusion fluid. In some implementations, the amount of nucleic acids is an amount of low molecular-weight DNA (e.g., less than 200 base pairs) in the perfusion fluid. In some implementations, the molecular weight of nucleic acids is an average molecular weight of nucleic acids in the perfusion fluid.

In some implementations, the isolated nucleic acids are cell-free DNA. The method may further include evaluating that the cell-free DNA is derived from apoptosis if the cell-free DNA has a molecular weight that is consistent with nucleosomal DNA. In some implementations, the isolated nucleic acids are cellular DNA. In some implementations, the isolated nucleic acids are RNA. The RNA may be RNA present in extracellular vesicles, such as exosomes and microvesicles. Vesicles captured from perfusion fluid may be lysed to release the RNA, which may then be purified in solution using techniques such as filtration, hybrid capture, size selection, or other suitable techniques. The method may further include the step of evaluating the amount of isolated RNA as a biomarker of stability or quality of the organ/tissue. The method may further include evaluating a gene expression characteristic corresponding to the isolated RNA to evaluate a cellular origin of the RNA. For example, the cellular origin of the RNA is evaluated to be an immune cell when the gene expression characteristic relates to immune function. RNA could be used as a biomarker, but may also provide a better picture of the origin when it is packaged in a vesicle (e.g., better signature of cell viability or status in organ), for example, compared to free RNA. Analysis of RNA may give more functional information regarding the status of an organ or tissue compared to analysis of DNA, due to the role of RNA in gene expression. Isolated RNA may be measured to evaluate at least one of a type of the RNA (e.g., coding or non-coding) or a sequence of the RNA (e.g., an mRNA sequence). The type and the sequence(s) may provide information indicative of an origin of the RNA that is found in the perfusion fluid and help inform about the status of the organ or tissue. Non-coding RNA can function as a modulator of expression, so presence or absence of non-coding RNA can be a predictor of transplant outcome and can be considered in addition to, or in alternative to DNA or coding RNA. Manipulation and analysis of RNA can be more difficult than that of DNA, due to the instability (due to the single-stranded form and degradation of uracil) of RNA and lower natural RNA concentration. Accordingly, additional techniques may be required to accurately analyze RNA from perfusate or flush samples. For example, more selective isolation and extraction methods may be needed to account for the lower concentration of RNA. RNA may be reverse transcribed to produce complementary DNA (cDNA) in order to solve the issue of instability.

The method may further include normalizing the amount of nucleic acids relative to at least one of a size, weight, volume, or surface area of the donor organ or donor tissue; perfusion time; perfusion volume; or temperature of the perfusion fluid. The method may include evaluating a fragmentation pattern of the nucleic acids. For example, the method may include categorizing the isolated nucleic acids into nucleic acids derived from random degradation of rupturing cells in the perfusion fluid and nucleic acids derived from apoptosis of cells in the donor organ or donor tissue. The method may further include obtaining one or more additional samples of the perfusion fluid taken from different points in time than the sample; quantifying an amount of nucleic acids (e.g., cellular DNA, cfDNA, or RNA) in the perfusion fluid of the one or more additional samples; and tracking the amount of nucleic acids in the fluid perfusion over time based on the sample and the one or more additional samples. The nucleic acids may include cellular DNA, cfDNA, RNA, or a combination thereof.

In a second aspect, provided herein is a method for predicting an outcome of a transplant, the method including preparing a preparation of nucleic acids according to the method of the first aspect above and further including hypothesizing a predicted outcome of transplantation of the donor organ or donor tissue based on the amount of nucleic acids.

In some implementations, the nucleic acids are cellular DNA from donor-derived cells, and the predicted outcome is a predicted favorable or unfavorable prognosis based on the cellular DNA, for example, the cellular DNA being indicative of an immune response. In some implementations, the nucleic acids are cell-free DNA, and the predicted outcome is a predicted favorable or unfavorable prognosis based on the cell-free DNA, for example, the cell-free DNA being indicative of damage to the donor organ or donor tissue. In some implementations, the predicted outcome is a predicted unfavorable prognosis being a decision maker for not using the organ for transplantation. In some implementations, the predicted outcome is hypothesized based at least in part on a risk call generated via an algorithm using the amount of nucleic acids as an algorithm input. In some implementations, the predicted outcome is hypothesized by performing a “quality” evaluation of the donor organ or donor tissue based on the amount of nucleic acids. Such evaluation may indicate at least one of the following characteristics associated with transplantation outcome: a presence of delayed graft function, a duration of delayed graft function, a rate of primary non-function, and an organ function at various time points after transplant.

In a third aspect, provided herein is a method for predicting an outcome of a transplant, the method including preparing a nucleic acid preparation according to the method of the first aspect above and further including hypothesizing a predicted outcome of transplantation of the donor organ or donor tissue based on the fragment size distribution (or a subset thereof) of the nucleic acids. In some implementations, the predicted outcome is hypothesized based at least in part on a risk call evaluated via an algorithm using the fragment size distribution (or a subset thereof) as an algorithm input.

In some implementations of either the second aspect or the third aspect, the predicted outcome is rejection of the transplantation or non-rejection of the transplant. The predicted outcome may include one or more of a type of rejection of the transplant and a time of rejection of the transplant. The method of either the second aspect or the third aspect may further include making a recommendation to cease perfusion of the organ or tissue based on the predicted outcome. The recommendation to cease perfusion may be made if the amount of nucleic acids analyzed in one or more preparations exceeds a threshold amount of nucleic acids (e.g., a threshold calculated based on the organ/tissue size).

In some implementations of any of the first aspect, second aspect, or third aspect, the method further includes performing a targeted genetic analysis of the isolated nucleic acids in one or more preparations to identify a genetic feature in the nucleic acids. Targeted genetic analysis may include targeted amplification and high-throughput sequencing of at least 50 target loci in the nucleic acids. The targeted genetic analysis may be used to evaluate a change in homeostasis or in cellular processes of the organ/tissue.

In a fourth aspect, provided herein is a method for feedback-controlled mechanical perfusion of a donor organ or donor tissue. The method includes perfusing a donor organ or donor tissue at a first value of a perfusion parameter in a perfusion chamber holding the donor organ or donor tissue; performing the method of any of the first aspect, second aspect, or third aspect on the donor organ or donor tissue; generating an appropriate adjustment to the perfusion parameter based at least in part on the amount of nucleic acids in the perfusion fluid; and adjusting the perfusion parameter to a second value based on the generated appropriate adjustment.

In some implementations, the adjustment is an increase or a decrease in a perfusion flow rate. In some implementations, the perfusion flow rate is decreased to a minimum threshold perfusion rate required to maintain the transplant in a state hypothesized or expected to have a threshold likelihood of transplant rejection. The threshold likelihood may be a maximum likelihood of transplant rejection. In some implementations, the perfusion parameter is a concentration of at least one component of the perfusion fluid. For example, the at least one component is selected from the group of oxygen, stem cells, an immunosuppressive drug, a nutrient, or red blood cells. In some implementations, the perfusion parameter is a temperature or a pH of the perfusion fluid.

The method may further include taking a post-adjustment sample of perfusion fluid; and evaluating whether an additional adjustment is necessary.

In any of the above aspects, the donor organ may be a kidney, a lung, a heart, a liver, a gallbladder, a pancreas, an intestine, or other organ. The donor tissue may be or include a heart valve, a skin tissue, a bone tissue, a tendon, a cornea, a blood vessel, a cartilage tissue, a ligament, an eye tissue, or a bone marrow tissue, or other tissue. The donor tissue may include blood, platelets, cord blood stem cells or peripheral blood stem cells. A person of skill in the art will recognize that any donor tissue or graft may be subject to the described and claimed methods, and that the above named donor tissues are exemplary and not limiting.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1 shows a block diagram of a machine perfusion system for storing and/or transporting a donor organ or tissue, according to an illustrative implementation;

FIG. 2 shows a flowchart describing a method for preparing a nucleic acid preparation from perfusate, according to an illustrative implementation;

FIG. 3 shows a flowchart describing a method for predicting an outcome of a transplant by perfusate nucleic acid analysis, according to an illustrative implementation;

FIG. 4 shows a flowchart describing a method for feedback-controlled mechanical perfusion of a donor organ or tissue, according to an illustrative implementation;

FIG. 5A shows a DNA size distribution of cell-free DNA isolated from perfusate samples, and FIG. 5B shows a DNA size distribution of cellular DNA isolated from perfusate samples;

FIG. 6A shows a DNA size distribution of cell-free DNA isolated from perfusate samples, FIG. 6B shows a DNA size distribution of cellular DNA isolated from perfusate samples, and FIG. 6C shows a DNA size distribution containing nucleosomal DNA of a particular sample;

FIG. 7 shows a plot of cellular DNA yield vs. cell-free DNA yield from perfusate samples;

FIG. 8 shows a chart of percentage cell-free DNA yield relative to kidney weight for each perfusate sample;

FIG. 9A shows a plot of cell-free DNA yield vs. perfusion time, and FIG. 9B shows a plot of cellular DNA yield vs. perfusion time.

### DETAILED DESCRIPTION

To provide an overall understanding of the systems, method, and devices described herein, certain illustrative embodiments will be described. Although the embodiments and features described herein are specifically described for use in connection with mechanical perfusion of organs and tissues, it will be understood that all the components and other features outlined below may be combined with one another in any suitable manner and may be adapted and applied to other types of transplantation and/or perfusion methods. For example, fluid samples may be taken from organs or tissues in static cold storage (SCS) or cryopreservation or from fluids used to flush organs or tissues prior to transplantation.

The systems, devices, and methods described herein allow for generating nucleic acid preparations from, and subsequent analysis of, nucleic acids sampled from perfusate, the fluid used for mechanical perfusion of an organ or tissue received from a donor, or from flush, the fluid used for preparing an organ or tissue prior to transplantation. While mechanical perfusion is used to preserve or re-condition the organ/tissue in storage or transportation prior to transplantation into a recipient, the perfusate may be sampled, according to the methods described herein, in order to isolate and analyze nucleic acids. Similarly, flush may be sampled and analyzed during or after preparation of the organ or tissue for transplantation. Nucleic acids, for example, cell-free DNA (cfDNA), cellular DNA, or RNA, from the perfusate may be quantified, enriched to generate unnatural preparations for purposes of analysis, sequenced, amplified to generate unnatural preparations for purposes of analysis, or otherwise manipulated to generate unnatural preparations for purposes of analysis in order to inform physicians as to a current status of the perfused organ/tissue. Analytical results, such as cfDNA concentration or fragment size distribution (or a subset thereof), may indicate that the organ/tissue has been injured and may no longer be suitable for transplantation or may require adjustments to the perfusion. The analytical results may also be indicative of a quality of the donor organ/tissue that can be used to assess its suitability for transplantation and for informing the process of allocating the organ/tissue to a suitable recipient. Levels of nucleic acids, for example, cfDNA, in the perfusate may be predictive of primary graft dysfunction after transplantation of the organ/tissue.

It should be understood that the systems and methods described herein can be applied to any organ, tissue, or biological entity that is perfused or fluidically treated prior to or during transplantation. The present disclosure is applicable to, but not limited to, a kidney, a lung, a heart, a liver, a gallbladder, a pancreas, an intestine, a heart valve, a skin tissue, a bone tissue, a tendon, a cornea, a blood vessel, a cartilage tissue, a ligament, an eye tissue, or a bone marrow tissue, or a combination thereof. In some implementations, the organ/tissue of interest is donated blood, donated platelets, cord blood stem cells, or peripheral blood stem cells, any of which may be stored in a fluid which can be sampled for nucleic acid analysis. These organs, tissues, or biological entities may be perfused and/or sampled before or after organ recovery from the donor. The term “graft” may be used to describe one or more of the organs or tissues described herein, and grafts suitable for these methods and systems include, but are not limited to, autografts, isografts, allografts, and xenografts.

While the examples in the present disclosure discuss various techniques for making DNA preparations, manipulation of DNA, and DNA analysis, it should be understood that a person of ordinary skill in the art may extend the present disclosure towards manipulation and analysis of RNA or any other nucleic acid present in perfusion fluid or flush. The examples provided herein may refer only to perfusate, but it should be understood that the methods may be applied to flush samples without departing from the scope of the disclosure. Both fluids, perfusate and flush, may be referred to as “graft fluids” herein. The graft fluids may contain an oxygen carrier, a buffer or priming solution, a colloid, nutrient supplementations, anticoagulants, protective additives, and/or antibiotics. The graft fluids may be red blood cell based solutions, acellular solutions, or whole blood. The oxygen carrier may comprise red blood cells. In some embodiments, the oxygen carrier is a hemoglobin-based oxygen carrier such as hemopure or breonics. Hemopure is a polymerized bovine hemoglobin-based oxygen carrier. Breonics is exsanguinous metabolic support system that contains a highly enriched tissue culture-like medium with amino acids, lipids, carbohydrates, and bovine hemoglobin. The acellular graft solution may be LIFOR®, which is a nonprotein oxygen carrier, Aqix RS-I®, which is mainly used to preserve tissue biopsies, and STEEN® solution, which has a high concentration of albumin and dextran. STEEN® solution may be diluted, particularly if used for kidney perfusion. The graft fluids may contain agents that prevent cellular edema such as albumin. The graft fluids may contain agents that increase osmolarity and/or enhance blood flow such as mannitol. The graft fluids may contain vasodilators such as prostacyclin. The prostacyclin may be synthetic such as epoprostenol. The vasodilators may also be verapamil or sodium nitroprusside. The graft fluids may contain agents that prevent inflammation such as corticosteroids. The graft fluids may contain nutrients such as glucose, amino acids, insulin, and/or multivitamins. The graft fluids may also contain antibiotics. The graft solutions may contain priming solutions such as RINGER'S® solution, STEEN® solution, Plasma Lyte A, or Williams media E. In one preferable embodiment, the graft fluid is a combination of STEEN® Solution as either acellular or with addition of packed RBC to the perfusate, optionally wherein autologous whole blood is added to the STEEN® instead of RBC. STEEN® solution composition: (1000 ml contains 5 g dextran 40 [0.125 mM], 70 g bovine serum albumin [1.05 mM], 5.03 g NaCl [85.90 mM], 0.24 g glucose monohydrate [1.21 mM], 0.34 g KCl [4.56 mM], 0.19 g NaH2PO4·2H2O [1.22 mM], 0.22 g CaCl2·2H2O [1.50 mM], 0.24 g MgCl2·6H2O [2.52 mM], 1.26 g NaHCO₃[15.00 mM], and adjustment to a pH 7.4 with 1M NaOH). Table 2 below includes further illustrative graft fluids disclosed in Elliott et al., Am. J. of Transpl. 21(4), 1382-1390 (2021), incorporated herein by reference.

TABLE 2
Illustrative graft solutions

| | Oxygen | Priming | Fluid | | Nutrient/ | | Protective | |
|---|---|---|---|---|---|---|---|---|
| Model | carrier | solution | replacement | Colloid | supplementation | Anticoagulant | additives | Antibiotics |
| Human | RBC 1 | Ringer's | Ringer's | | Glucose | Heparin | Mannitol 10%, | |
| clinical | unit | solution | solution | | 5%, | | dextamethasone 8 | |
| | | | | | insulin | | mg, epoprostenol | |
| | | | | | 100 IU, | | sodium (Flolan) | |
| | | | | | synthamin | | 0.5 mg, sodium | |
| | | | | | 17 | | bicarbonate | |
| | | | | | | | 8.4% b, | |
| | | | | | | | multivitamins | |
| | | | | | | | (cernevit) | |
| Human | — | STEEN | | STEEN | | | Calcium | Ampicillin 1 |
| clinical | | solution 1 L, | | solution | | | gluconate 7 ml, | g |
| | | Ringer's | | | | | sodium | |
| | | solution 1 L | | | | | bicarbonate 8.4% | |
| | | | | | | | 16 ml | |
| Human | RBC | Plasma Lyte | Plasma Lyte | | TPN | Heparin 2000 | Multivitamins 5 | |
| | 250 ml | A 250 ml | A | | baxter, | IU | ml, sodium | |
| | | | | | clinimix, | | bicarbonate 8.4% | |
| | | | | | insulin | | 26 ml/L | |
| | | | | | 100 IU | | | |
| Human | RBC 1 | 5% human | Ringer's | | TPN | | Mannitol 10% 10 | Cefuroxime |
| | unit | albumin 250 | solution/urine | | nutriflex | | ml, epoprostenol | 750 mg |
| | | ml | recirculation | | | | sodium 4 μg/h, | |
| | | | | | | | calcium gluconate | |
| | | | | | | | 10% 10 ml, | |
| | | | | | | | sodium | |
| | | | | | | | bicarbonate 8.4% | |
| | | | | | | | 5-15 ml | |
| Human | RBC/ | Williams | Ringer's | | Dextrose, | Heparin 1000 | Dextamethasone 8 | |
| | hemopure | media E | solution | | insulin 5 | U/L | mg/L, sodium | |
| | 500 | 1500 ml | | | U/L | | bicarbonate 8.4% | |
| | ml | | | | | | | |
| Porcine | RBC | Williams | | Albumin | | Heparin | | Amoxicillin- |
| | 350 ml | media E 500 | | (bovine | | | | Clavulanate |
| | | ml | | serum | | | | 1000 mg/200 |
| | | | | albumin) | | | | mg |
| | | | | 40 g | | | | |
| Porcine | RBC | STEEN | Ringer's | STEEN | Amino | Heparin 1000 | Verapamil 0.25 | |
| | 125 ml | solution 150 | solution | solution | acids 1 | IU | mg/h, calcium | |
| | | ml, Ringer's | | | ml/h, | | gluconate 10% | |
| | | solution 200 | | | glucose 1 | | 100 mg/ml, | |
| | | ml, Double | | | ml/h, | | sodium | |
| | | reverse | | | insulin 5 | | bicarbonate 8.4% | |
| | | osmosis | | | IU/h | | 8 ml | |
| | | water 27 ml | | | | | | |
| Porcine | RBC | Ringer's | | Albumin | Amino | Heparin 2000 | Dexamethasone | Ampicillin 1 |
| | 202 ml | solution 282 | | (bovine | acids | IU + 500 IU/h | 10 mg, verapamil | g, Cefotaxime |
| | | ml, water 44 | | serum | 0.05 g/h, | | 0.25 mg/h, | 1 g |
| | | ml, Krebs- | | albumin) | insulin 5 | | calcium gluconate | |
| | | Henseleit | | 22.5 g | U/h | | 10% 3 ml, sodium | |
| | | Buffer 322 | | | | | bicarbonate 8.4% | |
| | | ml | | | | | | |
| Porcine | Whole | | | | Dextrose, | Heparin 4000 | Methylprednisolone | Cefotaxime 1 |
| | blood/ | | | | amino | U + 500 U/h | 50 mg, | g |
| | STEEN | | | | acids, | | multivitamins | |
| | solution | | | | insulin | | 0.25 mg/h, 10% | |
| | | | | | 1.5 U/h | | calcium gluconate | |
| | | | | | | | (whole blood | |
| | | | | | | | only) 20 ml, | |
| | | | | | | | verapamil 10 | |
| | | | | | | | mg + 1 mg/h or | |
| | | | | | | | nitroprusside 25 | |
| | | | | | | | mg/h or | |
| | | | | | | | vasodilator | |
| | | | | | | | cocktail, sodium | |
| | | | | | | | bicarbonate 8.4% | |
| | | | | | | | 20 ml | |

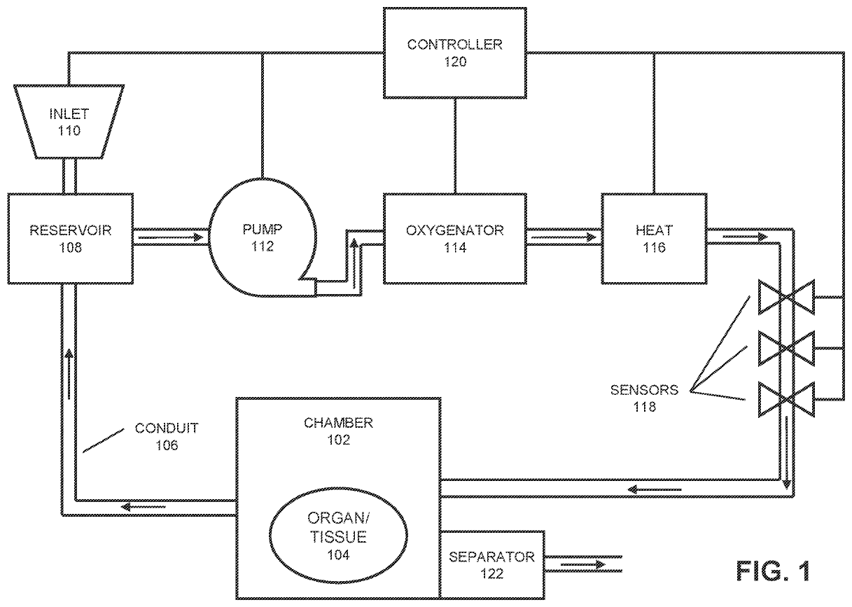

FIG. 1 shows an exemplary mechanical perfusion system 100 for storing, preserving, transporting, and/or re-conditioning a donor organ or donor tissue 104 for transplantation to a recipient. System 100 includes a chamber 102 for holding the organ/tissue 104. Perfusate is delivered into and out of chamber 102 via a conduit assembly 106 that fluidically connects chamber 102 to other components of system 100, such that the conduit assembly 106 and the components define a fluidic circuit. The components include a reservoir 108 for holding perfusate directed away from chamber 102. An inlet 110 is coupled to reservoir 108. The components further include a pump 112 for driving perfusate through the fluidic circuit via conduit assembly 106, an oxygenator 114 for supplying oxygen to the perfusate, a heat exchanger 116 for cooling or heating the perfusate, and one or more sensors 118 for measuring one or more properties of the perfusate. A separator 122 is provided in chamber 102 (or is otherwise operatively coupled to chamber 102) for removing one or more substances from the chamber or perfusate. A controller 120 is operatively coupled to each of inlet 110, pump 112, oxygenator 114, heat exchanger 116, and sensor(s) 118, such that controller 120 may send and/or receive data from each component.

Described in further detail below, methods for analysis of perfusate may be performed after sampling perfusate from system 100. The results of the analysis may be used to inform feedback control of system 100 or by a physician or operator to adjust certain parameters of system 100 manually. While the components of system 100 are depicted in a certain order connected in the fluidic circuit via conduit assembly 106, it is to be understood that the components may be rearranged in any suitable order. Two or more of the components may be arranged in series or in parallel, where in the latter configuration the conduit assembly splits into two or more flow paths for supplying perfusate through each of the two or more components. Certain components may be omitted.

It should be understood that organ/tissue 104 may be any biological entity that requires or benefits from perfusion, for example, for preservation, storage, transportation, and/or re-conditioning. Examples include a kidney, a lung, a heart, a liver, a gallbladder, a pancreas, an intestine, a heart valve, a skin tissue, a bone tissue, a tendon, a cornea, a blood vessel, a cartilage tissue, a ligament, an eye tissue, a bone marrow tissue, a volume of blood, a volume of platelets, a volume of cord blood stem cells, or a volume of peripheral blood stem cells.

Perfusate conveyed through the fluidic circuit of system 100 may be any perfusion solution used for preservation or preparation of a graft. For example, the perfusate may be or include a solvent, such as water, containing a combination of proteins, sugars, and/or soluble salts. In some implementations, the perfusate includes red blood cells. Perfusate may be a sterile, isotonic solution. For example, the KPS-1® Kidney Perfusion Solution (Organ Recovery Systems, Itasca, IL) may be used for machine perfusion of kidneys. A suitable perfusate may be or include sodium lactate solution (also known as Ringer's lactate solution or Hartmann's solution). Perfusate may contain at least one of sodium chloride, sodium lactate, potassium chloride, calcium chloride, magnesium sulfate, mannitol, dexamethasone, glutathione, or insulin. In any implementation, the perfusate within chamber 102 and the perfusate conveyed out of chamber 102 by conduit assembly 106 contain nucleic acids from organ/tissue 104. The nucleic acids in the perfusate may be present as cellular DNA, cell-free DNA, or RNA, or a combination thereof.

System 100 may include a housing such that one or more components are enclosed within the housing. For example, every component may be enclosed within the housing. The housing may include one or more ports for connection to external sources or drains, for example, for inlet 110 or separator 122. The housing may be a transporter configured with a carry handle for transporting and preserving donor organ/tissue 104. Alternatively, system 100 does not include a housing. The components may be loosely disposed and connected via conduit assembly 106 and electrical leads for controller 120. In some implementations, chamber 102 is at least part of a cadaver, and organ/tissue 104 has not yet been extracted from the cadaver. The rest of system 100 may be enclosed in a housing that is fluidically connected to the cadaver via conduit assembly 106.

Chamber 102 is a receptacle for aseptically holding organ/tissue 104 during storage or transport and generally for preservation and/or re-conditioning of organ/tissue 104. Chamber 102 may be a vessel having walls, an inlet, and an outlet, the inlet and outlet being fluidically coupled to conduit assembly 106 for conveying perfusate in and out of chamber 102. Chamber 102 may be a sterile and/or single-use vessel. Chamber 102 may be constructed from a biocompatible material, such as thermoplastic elastomer (TPE). Chamber 102 may include a lid that allows access of chamber 102 and organ/tissue 104. Chamber 102 may have a rigid, flexible, or collapsible construction. In some implementations, chamber 102 includes one or more measurement ports, and sensor(s) 118 may be inserted into chamber 102 via the one or more measurement ports for measuring characteristics of the perfusate, characteristics of organ/tissue 104, and/or conditions of chamber 102. Examples of measurements are discussed in further detail below.

Conduit assembly 106 may be formed of any suitable tubing material, such as a biocompatible plastic. Conduit assembly 106 may be rigid or flexible. In some implementations, electrical leads are attached to or formed on conduit assembly 106 for connection between controller 120 and any of pump 112, oxygenator 114, heat exchanger 116, or sensor(s) 118.

Reservoir 108 receives perfusate conveyed from chamber 102 via conduit assembly 106. Reservoir 108 may be a holding chamber with a perfusate inlet and a perfusate outlet each coupled to conduit assembly 106 for conveying perfusate. Inlet 110 is coupled to reservoir 108 for introduction of substances, such as new perfusate, new perfusate components, therapeutics, a nutrient supply, stem cells, and/or blood cells. Inlet 110 may be operatively coupled to controller 120, such that substances can be added to perfusate in response to user input to controller 120 or in a feedback-control loop implemented by controller 120, as discussed in further detail below. In some implementations, separator 122 is connected to or disposed in reservoir 108 and removes certain substances from the perfusate, as described in further detail below.

Pump 112 drives flow of perfusate through the fluidic circuit of system 100. Pump 112 may be any suitable type of fluid pump, such as a positive displacement (e.g., peristaltic, plunger, or piston) of non-positive displacement (e.g., centrifugal) pumps. Pump 112 may induce continuous or pulsatile flow of perfusate. Pump 112 is operatively coupled to controller 120, which may utilize a user input or use feedback control to set a flow rate of pump 112. The flow rate may be set on a per-mass basis for organ/tissue 104.

Oxygenator 114 supplies oxygen to the perfusate. Oxygenator 114 may include a port for receiving oxygen from an external source such as an oxygen concentrator, an oxygen tank, or wall oxygen. Alternatively, oxygenator 114 may include an oxygen tank or an oxygen concentrator. For example, oxygenator 114 is an oxygen concentrator and includes an air intake for receiving ambient air which is then concentrated to produce a flow of oxygen-enriched air for oxygenation of the perfusate.

Heat exchanger 116 is configured to cool or heat the perfusate. Perfusion of organ/tissue 104 using system 100 may be performed at different temperatures, ranging from hypothermic (4 to 10° C.) to sub-normothermic (15 to 30° C.) and to normothermic (about 37° C.). Accordingly, heat exchanger 116 may be used to maintain a target temperature (e.g., between 0 and 40° C.) of the perfusate by heating or cooling. Controller 120 is operatively coupled to heat exchanger 116 to set a target temperature and/or adjust the temperature of perfusate. Controller 120 may take user inputs to set or adjust a target temperature, and heat exchanger 116 is then controlled to reach the target temperature. In some implementations, a feedback control method is used to adjust the perfusate temperature. Heat exchanger 116 may include a temperature sensor, or sensor(s) 118 may measure temperature and communicate the measured temperature to controller 120 for appropriate control of heat exchanger 116.

Sensor(s) 118 are configured to measure one or more properties of the perfusate. Properties measured by sensor(s) 118 may include at least one of temperature, flow rate, pH, oxygen concentration, nucleic acid concentration, toxin concentration, or concentration of another perfusate component. Values of measured properties are communicated by sensor(s) 118 to controller 120. Measured values may be used for feedback control of one or more components of system 100 or for informing clinical decision-making.

Separator 122 may be used to remove substances from chamber 102. While it is shown in FIG. 1 that separator 122 is adjacent or attached to chamber 102, it should be understood that the separator or outlet may be provided at any point in the fluidic circuit of system 100, for example, in conduit assembly 106 or in reservoir 108. For example, separator 122 is a filter (e.g., micro-pore filter, ultrafiltration device), a separation column, a settled-substance trap, or collection of probes (e.g., affinity probes, magnetic probes). Substances removed by separator 122, such as metabolites, may be flushed from system 100.

Controller 120 is, for example, a microcontroller, processor, or printed circuit board, configured for receiving data or user inputs and controlling various components of system 100. Controller 120 may implement adaptive feedback control of the perfusion based on measured properties of the perfusion, or by receiving inputs characteristic of stability or injury of organ/tissue 104. Such inputs may be results generated according to the analytical methods described herein.

### Perfusate Sample Preparation Methods

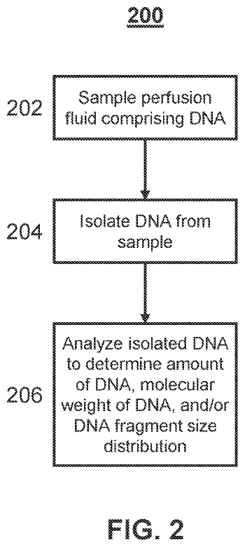

As discussed above, perfusate from a mechanical perfusion system, such as system 100, may be sampled, and nucleic acids in the perfusate sample may manipulated, amplified, prepared, and/or analyzed according to the methods described herein. FIGS. 2-4 describe methods for performing and utilizing this nucleic acid analysis.

FIG. 2 shows a flowchart describing an exemplary method 200 for preparing a nucleic acid sample from perfusion fluid used in preservation and/or re-conditioning of a donor organ/tissue. This method is particularly useful for evaluating or predicting the quality of the donor organ/tissue for transplantation and subsequent transplantation outcome of the donor organ/tissue. Method 200 includes steps 202, 204, and 206. Step 202 involves obtaining a sample of perfusion fluid from a donor organ/tissue or a machine perfusion system, such as system 100 described above in relation to FIG. 1. The sample includes nucleic acids. Step 204 involves isolating the nucleic acids from the sample. Step 206 involves performing an analysis of the isolated DNA preparation to evaluate at least one of: an amount of nucleic acids in the perfusion fluid, a molecular weight of nucleic acids in the perfusion fluid, or a fragment size distribution of nucleic acids in the perfusion fluid.

Nucleic acids isolated from the sample in step 204 may be cellular DNA, cell-free DNA (cfDNA), or RNA. Accordingly, the cellular DNA, cfDNA, or RNA may be separated from the perfusate in the sample using isolation techniques including, but not limited to, centrifugation, size selection, hybrid capture, or other suitable techniques. In some embodiments, the sample may be centrifuged to separate various layers. In some embodiments, the nucleic acids may be isolated using filtration. In some embodiments, the preparation of the nucleic acids may involve amplification, separation, purification by chromatography, liquid-liquid separation, preferential enrichment, preferential amplification, targeted amplification, reverse transcription, or any of a number of other techniques described herein, or any combination thereof. In some embodiments for the isolation of DNA, RNase is used to degrade RNA. Alternatively, DNase may be used to degrade DNA for isolation of RNA. Suitable purification techniques further include, but are not limited to: differential ultracentrifugation, density-gradient ultracentrifugation, polymer-facilitated precipitation, immunoaffinity capture, and size-exclusion chromatography. A person having ordinary skill in the art will recognize that the foregoing isolation techniques are exemplary and not exhaustive. The foregoing or other isolation techniques may be integrated with measurement, quantification, and multi-omics characterization techniques on a microfluidic platform.

For example, centrifugation may be used to separate cell pellets from the perfusion fluid. Cellular DNA may be purified from cellular pellets using methods or devices, such as (but not limited to) cellular DNA-specific probes, size selection, or a selectively-binding silica-based membranes, and cfDNA may be purified from the remaining perfusion fluid by methods or devices, such as (but not limited to) cfDNA-specific probes, size selection, or a circularizing DNA system. The nucleic acids may be isolated from the cell sources by a variety of extraction methods. Such methods may involve lysing the cell, thereby liberating nucleic acids so as to leave chromatin structure sufficiently intact to allow the preparation nucleosomal ladders, i.e., nucleosomal preparations. Suitable cell lysis methods include methods in which the nucleus is separately released for subsequent isolation and methods in which the nuclear membrane is dissolved. In some embodiments, the cells may be permeabilized, e.g., using a detergent such as lysolecithin, so as to retain chromatin structure. In some embodiments, the cell membrane may be disrupted by inducing apoptosis in the cells of the cell source. It is of interest to prepare nucleic acids that are of free of other cellular components so as to enable the biochemical manipulation of the nucleosomal ladders for use in subsequent procedures, e.g., DNA sequencing. In an embodiment, nucleic acid systems can be used to purify cellular DNA. For example, cfDNA may be isolated from the remaining perfusion fluid using beads having a specialized surface chemistry.

Techniques such as amplification may be used between steps 204 and 206 to further prepare, modify, purify, and/or enrich the isolated nucleic acids. In some embodiments, universal tagged adaptors are added to produce a library. In some implementations, tagged adaptors can be added using PCR. In some implementations, tagged adaptors can be added using ligation. Prior to ligation, sample nucleic acids may be blunt ended, and then a single adenosine base is added to the 3-prime end. Prior to ligation the nucleic acids may be cleaved using a restriction enzyme or some other cleavage method. During ligation, the 3-prime adenosine of the sample fragments and the complementary 3-prime tyrosine overhang of adaptor can enhance ligation efficiency. In some embodiments, the library is amplified using universal primers. In an embodiment, the amplified library is fractionated by size separation or other methods. In some embodiments, PCR amplification is used to amplify target loci. In some embodiments, the amplified nucleic acids are sequenced (such as, for example, sequencing using an ILLUMINA IIGAX or HiSeq sequencer). In some embodiments, the amplified nucleic acids are sequenced from each end of the amplified nucleic acids to reduce sequencing errors. If there is a sequence error in a particular base when sequencing from one end of the amplified nucleic acids, there is less likely to be a sequence error in the complementary base when sequencing from the other side of the amplified nucleic acids (compared to sequencing multiple times from the same end of the amplified nucleic acids). Accordingly, the amplified nucleic acids may be re-sequenced (or the sequencing cycles may be increased) from one or both ends to increase the “depth-of-read”. The term “depth of read,” as used herein, refers to the number of sequencing reads that map to a given locus. The depth of read may be normalized over the total number of reads. When “depth of read” refers to a sample, it may mean the average depth of read over the targeted loci. When “depth of read” refers to a locus, it may refer to the number of reads measured by the sequencer mapping to that locus. In general, the greater the depth of read of a locus, the closer the ratio of alleles at the locus will tend to be to the ratio of alleles in the original sample of DNA. Generally, increasing depth-of-read may reduce errors in sequencing.

In some embodiments, whole genome application (WGA) is used to amplify a nucleic acid sample. In one embodiment, WGA is performed using ligation-mediated PCR (LM-PCR), in which short DNA sequences called adapters are ligated to blunt ends of DNA. These adapters contain universal amplification sequences, which are used to amplify the DNA by PCR. In another embodiment, WGA is performed using degenerate oligonucleotide primer PCR (DOP-PCR), in which random primers that also contain universal amplification sequences are used in a first round of annealing and PCR. Then, a second round of PCR is used to amplify the sequences further with the universal primer sequences. In another embodiment, WGA is performed using multiple displacement amplification (MDA), which uses the phi-29 polymerase, which is a highly processive and non-specific enzyme that replicates DNA and has been used for single-cell analysis. In some embodiments, WGA is not performed.

In some embodiments, selective amplification or enrichment are used to amplify or enrich target loci. In some embodiments, the amplification and/or selective enrichment technique may involve PCR such as ligation mediated PCR, fragment capture by hybridization, Molecular Inversion Probes, or other circularizing probes. In some embodiments, real-time quantitative PCR (RT-qPCR), digital PCR, droplet PCR, or emulsion PCR, single allele base extension reaction followed by mass spectrometry are used (Hung et al., J Clin Pathol 62:308-313, 2009, which is hereby incorporated by reference in its entirety). In some embodiments, capture by hybridization with hybrid capture probes is used to preferentially enrich the nucleic acids. In some embodiments, methods for amplification or selective enrichment may involve using probes where, upon correct hybridization to the target sequence, the 3-prime end or 5-prime end of a nucleotide probe is separated from the polymorphic site of a polymorphic allele by a small number of nucleotides. This separation reduces preferential amplification of one allele, termed allele bias. This is an improvement over methods that involve using probes where the 3-prime end or 5-prime end of a correctly hybridized probe are directly adjacent to or very near to the polymorphic site of an allele. In an embodiment, probes in which the hybridizing region may or certainly contains a polymorphic site are excluded. Polymorphic sites at the site of hybridization can cause unequal hybridization or inhibit hybridization altogether in some alleles, resulting in preferential amplification of certain alleles. These embodiments are improvements over other methods that involve targeted amplification and/or selective enrichment in that they better preserve the original allele frequencies of the sample at each polymorphic locus, whether the sample is pure genomic sample from a single individual or mixture of individuals

In some embodiments, a PCR technique referred to as mini-PCR is used to generate very short amplicons (U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, filed Nov. 18, 2011, and U.S. Ser. No. 61/994,791, filed May 16, 2014, each of which is hereby incorporated by reference in its entirety). cfDNA is highly fragmented. For some cfDNA, the fragment sizes are distributed in approximately a Gaussian fashion with a mean of 160 bp, a standard deviation of 15 bp, a minimum size of about 100 bp, and a maximum size of about 220 bp. The polymorphic site of one particular target locus may occupy any position from the start to the end among the various fragments originating from that locus. Because cfDNA fragments are short, the likelihood of both primer sites being present the likelihood of a fragment of length L comprising both the forward and reverse primers sites is the ratio of the length of the amplicon to the length of the fragment. Under ideal conditions, assays in which the amplicon is 45, 50, 55, 60, 65, or 70 bp will successfully amplify from 72%, 69%, 66%, 63%, 59%, or 56%, respectively, of available template fragment molecules. In certain embodiments, the cfDNA is amplified using primers that yield a maximum amplicon length of 85, 80, 75 or 70 bp, and in certain preferred embodiments 75 bp, and that have a melting temperature between 50 and 65° C., and in certain preferred embodiments, between 54-60.5° C. The amplicon length is the distance between the 5-prime ends of the forward and reverse priming sites. Amplicon length that is shorter than typically used may result in more efficient measurements of the desired polymorphic loci by only requiring short sequence reads. In an embodiment, a substantial fraction of the amplicons are less than 100 bp, less than 90 bp, less than 80 bp, less than 70 bp, less than 65 bp, less than 60 bp, less than 55 bp, less than 50 bp, or less than 45 bp.

In some embodiments, amplification is performed using direct multiplexed PCR, sequential PCR, nested PCR, doubly nested PCR, one-and-a-half sided nested PCR, fully nested PCR, one sided fully nested PCR, one-sided nested PCR. hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, which are hereby incorporated by reference in their entireties. If desired, any of these methods can be used for mini-PCR.

If desired, the extension step of the PCR amplification may be limited from a time standpoint to reduce amplification from fragments longer than 200 nucleotides, 300 nucleotides, 400 nucleotides, 500 nucleotides or 1,000 nucleotides. This may result in the enrichment of fragmented or shorter DNA (such as DNA from cells that have undergone apoptosis or necrosis) and improvement of test performance.

In some embodiments, multiplex PCR is used. In some embodiments, the method of amplifying target loci in a nucleic acid sample involves (i) contacting the nucleic acid sample with a library of primers that simultaneously hybridize to least 100; 200; 500; 750; 1,000; 2,000; 5,000; 7,500; 10,000; 20,000; 25,000; 30,000; 40,000; 50,000; 75,000; or 100,000 different target loci to produce a reaction mixture; and (ii) subjecting the reaction mixture to primer extension reaction conditions (such as PCR conditions) to produce amplified products that include target amplicons. In some embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some embodiments, the primers are in solution (such as being dissolved in the liquid phase rather than in a solid phase). In some embodiments, the primers are in solution and are not immobilized on a solid support. In some embodiments, the primers are not part of a microarray. In some embodiments, the primers do not include molecular inversion probes (MIPs).

Step 206 involves performing an analysis of the isolated nucleic acid preparations to evaluate at least one of: an amount of nucleic acids in the perfusion fluid, a molecular weight of nucleic acids in the perfusion fluid, or a fragment size distribution of nucleic acids in the perfusion fluid. Various techniques may be used for performing this analysis. An appropriate technique may be selected by balancing variations in accuracy, reproducibility, sensitivity, labor intensity, speed, and cost between various techniques. Suitable techniques include, for example, using the NANODROP 1000 (THERMO SCIENTIFIC), QUBIT 2.0 HIGH SENSITIVITY ASSAY (LIFE TECHNOLOGIES), BIOANALYZER 2100 HIGH SENSITIVITY ASSAY (AGILENT TECHNOLOGIES), TAPESTATION 2200 HIGH SENSITIVITY D1000 KIT (AGILENT TECHNOLOGIES), GX TOUCH 24 HIGH SENSITIVITY ASSAY (PERKINELMER), or FRAGMENT ANALYZER HIGH SENSITIVITY NGS KIT (ADVANCED ANALYTICAL).

Suitable methods/techniques include but are not limited to UV-vis spectrophotometry, fluorimetry (e.g., fluorescent dye-based method, such as fluorescent in situ hybridization (FISH)), microfluidics, electrophoresis, automated electrophoresis, capillary electrophoresis, and droplet-based methods. In some embodiments, qPCR is used to measure cellular DNA, cfDNA, or RNA. For example, one or more loci from cfDNA or cellular DNA (such as Glyceraldehyd-3-phosphat-dehydrogenase, GAPDH) can be measured using multiplex qPCR. In some embodiments, fluorescence-labelled PCR is used to measure cfDNA, cellular DNA, or RNA. If desired, the normality distribution of the data can be calculated using methods, such as, but not limited to, the Shapiro-Wilk-Test. If desired, cfDNA, cellular DNA, or RNA levels can be compared using methods, such as the Mann-Whitney-U-Test. In some embodiments, cfDNA, cellular DNA, or RNA levels are compared with other prognostic factors using methods such as the Mann-Whitney-U-Test or the Kruskal-Wallis-Test.

The amount of nucleic acid evaluated in step 206 may be a total amount of nucleic acids in the perfusion fluid sample, an estimate of the total amount of nucleic acids in perfusate of a machine perfusion system, a concentration of nucleic acids in the perfusion fluid sample, an amount of low molecular weight DNA in the sample (e.g., fragments of less than 200 base pairs), or an amount of high molecular weight DNA in the sample (e.g., fragments of greater than or equal to 200 base pairs). The molecular weight of nucleic acids evaluated in step 206 may be an average molecular weight of nucleic acids in the perfusion fluid or a molecular weight distribution of the nucleic acids.

Method 200 may include additional steps for interpreting or refining the analytical results of step 206. For example, when the isolated and analyzed nucleic acids are cfDNA, method 200 may further include evaluating that the cfDNA is derived from apoptosis if the cfDNA has a molecular weight (evaluated in step 206) that is consistent with nucleosomal DNA. The method may further involve evaluating a change in homeostasis or in cellular processes based on the analytical results. For example, an elevated level of cfDNA in the perfusion fluid may indicate that the organ/tissue has trended away from homeostatis and may undergo or has undergone failure.

In some implementations, the nucleic acids isolated in the preparation are RNA. The RNA may be RNA present in extracellular vesicles, such as exosomes and microvesicles. Vesicles captured from perfusion fluid may be lysed to release the RNA, which may then be purified in solution using techniques such as filtration, size selection, or hybrid capture. The method may further include evaluating the amount of isolated RNA as a biomarker of stability or quality of the organ/tissue. The method may further include evaluating a gene expression characteristic corresponding to the isolated RNA to evaluate a cellular origin of the RNA. For example, the cellular origin of the RNA is evaluated to be an immune cell when the gene expression characteristic relates to immune function. RNA could be used as a biomarker like DNA, but may also provide a better picture of the origin when it is packaged in a vesicle (i.e., better signature of cell viability or status in organ), for example, compared to free RNA. Analysis of RNA may give more functional information regarding the status of an organ or tissue compared to analysis of DNA, due to the role of RNA in gene expression. Manipulation and analysis of RNA can be more difficult than that of DNA, due to the instability (due to the single-stranded form, degradation of uracil, and ubiquitous presence of ribonuclease enzymes in cells and tissues which rapidly degrade RNA) of RNA and lower natural RNA concentration. Accordingly, additional techniques may be desired to accurately isolate and analyze RNA from perfusate or flush samples. For example, more selective isolation and extraction methods may be used to account for the lower concentration of RNA. RNA may be reverse transcribed to produce complementary DNA (cDNA) in order to improve stability.

In order to account for the instability and low concentration of RNA, suitable techniques for isolation of the RNA from the sample or preparation include (but are not limited to) guanidinium-acid-phenol extraction, filter technology (e.g., glass fiber filters), density gradient centrifugation (e.g., using cesium chloride or cesium trifluoroacetate), magnetic bead technology (e.g., hybrid capture with biotin-labeled probes and streptavidin-coated magnetic beads), lithium chloride and urea isolation, chromatography (e.g., oligo(dt)-cellulose column chromatography), and non-column poly (A)+ purification/isolation. Suitable techniques may involve cell lysis and dissolution, denaturation of DNA and proteins, denaturation and inactivation of RNases, removal or separation of cellular components, or precipitation. In some embodiments, the RNA is reverse transcribed to produce cDNA, which is then analyzed in step 206. The results of the cDNA analysis may be traced back to characterize the RNA in the original sample or preparation.

In some implementations, the analysis of RNA (e.g., mRNA) from perfusate or flush is used to evaluate the cellular transcriptome(s) of the graft at any given moment. Accordingly, the method may involve transcriptome assembly from sequencing reads (e.g., by microarray or RNA-Seq) of the RNA or corresponding cDNA. In the case of cDNA, suitable sequencing techniques include, but are not limited to, next-gen sequencing (high throughput sequencing), shotgun sequencing, Sanger sequencing, pyrosequencing, or nanopore sequencing. Transcriptome assembly may be performed de novo (without a reference genome), such that prior genotyping of the graft or donor is not required, or genome guided. The de novo approach involves identifying contiguous sequences in the sequence reads (e.g., by using de Bruijn graphs). The genome guided approach may be used when the genome of the graft or donor is already known. Alternatively, DNA in the same or another perfusate or flush sample may be genotyped alongside the RNA analysis in order to produce a reference genome in parallel. A genome guided approach aligns sequence reads over contiguous sequences as well as non-contiguous sequences.

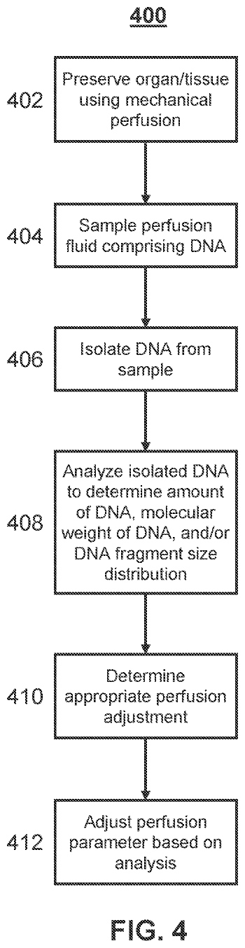

RNA analysis may also involve gene expression quantification. Quantification of expression can be useful to study changes in response to external stimuli, differences between healthy and diseased states, and other questions regarding graft stability. For example, changes in perfusion (or flush) conditions (e.g., temperature, pH, solute concentrations) may cause changes in gene expression within the graft cells, and analysis of this dynamic expression may be used to inform adjustments to the perfusion (or flush) conditions, for example, as described below with respect to FIG. 4. Expression may be quantified by counting the number of reads that mapped to each genetic locus in the transcriptome assembly step. Expression may be quantified for exons or genes, for example, using identified contiguities or reference transcript annotations. Observed read counts can be converted to appropriate metrics for hypothesis testing, regressions, or other analyses, for example, taking into account the sequence depth or coverage, gene length, total sample RNA (e.g., as evaluated in step 206), or variance in expression of each gene. Taking into account these parameters can help to normalize the results between samples and loci and decrease errors (e.g., sampling errors that propagate through the analysis).

As discussed herein, multiple samples (of perfusate or flush) may be taken over time or for different grafts, and made subject to method 200. RNA analysis from multiple samples can be especially useful for evaluating differential expression, whereby expression varies between two or more conditions (e.g., perfusion conditions such as temperature or pH) or between two or more candidate grafts (e.g., from the same donor or cadaver, or from donors have the same or similar genomes). One or more samples are taken for each condition or candidate grafts. Outputs of a differential expression analysis performed on the multiple samples include differentially expressed genes (DEGs), which can be up- or down-regulated. The differential expression analysis may take as inputs: (1) a gene expression matrix, including M genes for each of N samples that have been analyzed via gene expression quantification; and (2) a design matrix containing experimental conditions for the N samples. Relevant conditions include (but are not limited to) physical conditions (e.g., temperature, pH, or concentration of the perfusate or flush; graft size), batch effects (e.g., laboratory conditions, measurement errors, instruments or techniques used, reagent lot or batch, personnel differences, time of day), genetic effects (e.g., known artifacts or variations), and/or any metadata that might change gene expression. Conditions may be known or unknown, where unknown conditions can be estimated via machine learning approaches (e.g., principal component, surrogate variable). Hidden variable analyses may also be performed to identify conditions not already captured. The differential expression analysis may methodologically involve regression or non-parametric statistics to identify DEGs. Adjustments, such as familywise error rate or false discovery rate, may be employed to account for multiple hypotheses. Outputs of the differential expression analysis can include a table showing, for each gene, the log fold change, the p-value, and/or p-value adjusted for multiple comparisons. Log fold change cutoffs may be set to identify biologically relevant DEGs (those which pass the cutoffs and are statistically significant). Identified biologically relevant DEGs may be used to inform decision-making regarding the graft(s). For example, if immune response-associated genes are up-regulated, the graft may require perfusion adjustment or treatment with a drug (e.g., an immune stabilizer). Certain genes may be identified as biomarkers of graft rejection or failure, so identified up-regulation of those genes may be an indicator for predicting graft rejection or failure.

The results of step 206 may be normalized in order to account for certain parameters of the machine perfusion system from which the sample is taken. For example, the amount of nucleic acids, molecular weight, or fragment size distribution is normalized with respect to one or more of a perfusion time (period of time that the organ/tissue has been perfused), perfusion fluid volume, organ/tissue size, organ/tissue, weight, organ/tissue volume, organ/tissue surface area, perfusion fluid temperature, and perfusion fluid pH. Accordingly, each of these parameters may be measured prior to or during method 200.

Method 200 may further include a step of evaluating a fragmentation pattern of the nucleic acids. This fragmentation pattern may then be used to categorize the isolated nucleic acids into nucleic acids derived from random degradation of rupturing cells in the perfusion fluid and nucleic acids derived from apoptosis of cells in the donor organ/tissue.

Additional samples of perfusion fluid from the donor organ/tissue may be taken at different points in time. By repeating steps 204 and 206 on the additional samples, the analytical results such as amount of nucleic acids or molecular weight of nucleic acids may be tracked over a period of time. By monitoring the amount of nucleic acids, molecular weight, or fragment size distribution (or a subset thereof) over time, physicians may be able to closely monitor the quality or status of the donor organ/tissue and evaluate whether intervention or adjustment is required, as discussed in further detail below.

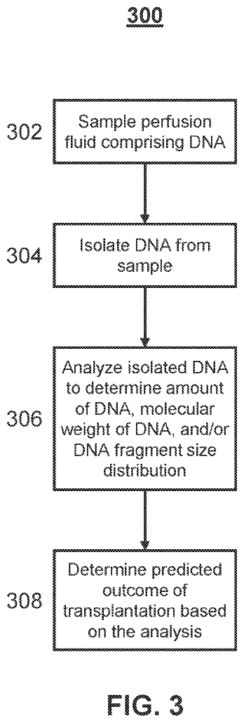

FIG. 3 shows a flowchart describing a method 300 for predicting an outcome of a transplant. Method 300 includes steps 302, 304, 306, and 308. Step 302 involves obtaining a sample of perfusion fluid from a donor organ/tissue or a machine perfusion system, such as system 100 described above in relation to FIG. 1. The sample includes nucleic acids. Step 304 involves isolating the nucleic acids from the sample to create an artificial preparation of nucleic acids. Step 306 involves performing an analysis of the preparation of isolated nucleic acids to evaluate at least one of: an amount of nucleic acids in the perfusion fluid, a molecular weight of nucleic acids in the perfusion fluid, or a fragment size distribution of nucleic acids in the perfusion fluid. Steps 302, 304, and 306 may be performed in the same ways as described above in relation to steps 202, 204, and 206, respectively, of FIG. 2. Step 308 involves predicting an outcome of transplantation based on the analysis performed in step 306, i.e., based on at least one of the amount of nucleic acids, the molecular weight of nucleic acids, or the fragment size distribution (or a subset thereof) of nucleic acids.

Predicted outcomes may include presence of delayed graft function, duration of delayed graft function, rate of primary non-function, organ function at various time points post-transplant, likelihood of transplant rejection, likelihood of transplant non-rejection, a type of transplant rejection, or a time of rejection.

The results of the analysis of step 306 can be informative of quality or status of a donor organ or tissue or of clinical outcomes of transplantation of said organ or tissue into a recipient. For example, when the isolated and analyzed preparation of nucleic acids are cellular DNA from donor-derived cells, the predicted outcome of step 308 may be a predicted favorable or unfavorable prognosis based on the cellular DNA being indicative of an immune response by the donor-derived cells of the organ/tissue. As another example, the isolated and analyzed nucleic acids are cfDNA, and the predicted outcome is a predicted favorable or unfavorable prognosis based on the cfDNA being indicative of damage to the donor organ/tissue. The predicted outcome of step 308 may be used as a decision maker for adjusting perfusion parameters or to not use the organ/tissue for transplantation, for example, if the predicted outcome is a predicted favorable or unfavorable prognosis. The predicted outcome may be used to make a recommendation to adjust or cease perfusion of the organ/tissue. For example, a recommendation to cease perfusion is made when an evaluated amount of nucleic acids in the perfusion fluid exceeds a threshold amount of nucleic acids.

The outcome of step 308 may be predicted based at least in part on a risk call generated via an algorithm using the evaluated amount of nucleic acids, molecular weight, or fragment size distribution (or a subset thereof) as an algorithm input(s). The outcome may be predicted by performing a quality evaluation of the donor organ/tissue based on the amount of nucleic acids, molecular weight, or fragment size distribution (or a subset thereof). Various methods may be used for performing the quality evaluation. For example, a modified kidney donor risk index may be calculated with incorporation of the evaluated amount of nucleic acids, molecular weight, or fragment size distribution (or a subset thereof), in order to summarize a risk of kidney graft failure. The quality evaluation may involve scoring the graft based on the nucleic acid analysis. Scoring includes, but is not limited to, calculating a graft-specific quality index (e.g., a liver graft quality index, lung graft quality index, or kidney donor risk index (KDPI)).

FIG. 4 shows a flowchart describing a method 400 for feedback-controlled mechanical perfusion of a donor organ or tissue. Method 400 includes steps 402, 404, 406, 408, and 410. Step 402 involves preserving a donor organ or tissue by mechanical perfusion at a first value of a perfusion parameter. Step 404 involves obtaining a sample of perfusion fluid from the donor organ/tissue or a machine perfusion system used for preservation in step 402, such as system 100 described above in relation to FIG. 1. The sample comprises nucleic acids. Step 406 involves isolating the nucleic acids from the sample. Step 408 involves performing an analysis of the isolated nucleic acids to evaluate at least one of: an amount of nucleic acids in the perfusion fluid, a molecular weight of nucleic acids in the perfusion fluid, or a fragment size distribution of nucleic acids in the perfusion fluid. Steps 404, 406, and 408 may be performed in accordance with any of the implementations described above in relation to steps 202/302, 204/304, and 206/306, respectively, of FIGS. 2 and 3. Step 410 involves generating an appropriate adjustment to be made to the perfusion parameter of the mechanical perfusion based at least in part on the results of the analysis (e.g., based on at least one of the amount of nucleic acids, the molecular weight, or the fragment size distribution (or a subset thereof)). Step 412 involves adjusting the perfusion parameter of the mechanical perfusion to a second value based on the generated appropriate adjustment.

Steps 410 and 412 may be performed in an automated fashion by a controller, such as controller 120 described above in relation to FIG. 1. The controller may include machine-readable instructions for performing feedback control of the perfusion parameter. Multiple perfusion parameters may be feedback-controlled in concert. The controller may use, but is not limited to, proportional control, proportional-integrative control, or proportional-integrative-derivative (PID) control. Feedback control may be performed responsive to a user input of the analytical results from step 408. Alternatively, the mechanical perfusion may be performed on a system allowing for user control of the perfusion parameter, and adjustment of the perfusion parameter may be performed manually by a physician/user.

The perfusion parameter may include but is not limited to: a perfusate flow rate, a perfusate temperature, a perfusate pH, an oxygen concentration of the perfusate, a concentration of a therapeutic drug (e.g., an immunosuppressive or anti-inflammatory drug, stem cells), a concentration of a perfusate component (e.g., salts, proteins, nutrients), or a perfusion duration. The appropriate adjustment may include a recommendation to adjust more than one perfusion parameter simultaneously or in succession.

In some implementations, method 400 further comprises the steps of taking a post-adjustment sample of perfusion fluid, and evaluating whether an additional adjustment is necessary. Any number of iterations of the steps of adjustment and re-evaluation of additional adjustment may be performed.

### Further Techniques

Methods 200, 300, and/or 400 may further include, at any point after isolating nucleic acids from the perfusion fluid sample, performing a targeted genetic analysis of the isolated nucleic acids to identify one or more genetic features in the nucleic acids. Amplification and sequencing (e.g., high throughput sequencing, microarray, nanopore sequencing) may be used in order to perform this targeted genetic analysis.

In some embodiments, the method includes isolating or purifying the nucleic acids. There are a number of procedures to accomplish such an end. In some embodiments, the sample may be centrifuged to separate various layers. In some embodiments, the nucleic acids may be isolated using filtration. In some embodiments, the preparation of the nucleic acids may involve amplification, separation, purification by chromatography, liquid-liquid separation, isolation, preferential enrichment, preferential amplification, targeted amplification, reverse transcription, or any combination thereof, or any of a number of other techniques described herein. In some embodiments for the isolation of DNA, RNase is used to degrade RNA. Alternatively, DNase may be used to degrade DNA to isolate RNA in the sample.

In some embodiments, universal tagged adaptors are added to produce a library. Prior to ligation, sample nucleic acids may be blunt ended, and then a single adenosine base is added to the 3-prime end. In some implementations, tagged adaptors can be added using PCR. In some implementations, tagged adaptors can be added using ligation. Prior to ligation the nucleic acids may be cleaved using a restriction enzyme or some other cleavage method. During ligation the 3-prime adenosine of the sample fragments and the complementary 3-prime tyrosine overhang of adaptor can enhance ligation efficiency. In some embodiments, the library is amplified using universal primers. In an embodiment, the amplified library is fractionated by size separation or other methods. In some embodiments, PCR amplification is used to amplify target loci. In some embodiments, the amplified nucleic acids are sequenced (such as sequencing using an ILLUMINA IIGAX or HiSeq sequencer). In some embodiments, the amplified nucleic acids are sequenced from each end of the amplified nucleic acids to reduce sequencing errors. If there is a sequence error in a particular base when sequencing from one end of the amplified nucleic acids, there is less likely to be a sequence error in the complementary base when sequencing from the other side of the amplified nucleic acids (compared to sequencing multiple times from the same end of the amplified nucleic acids).

In some embodiments, whole genome application (WGA) is used to amplify a nucleic acid sample. There are a number of methods that may be used for WGA: ligation-mediated PCR (LM-PCR), in which short DNA sequences called adapters are ligated to blunt ends of DNA. These adapters contain universal amplification sequences, which are used to amplify the DNA by PCR. In another embodiment, WGA is performed using degenerate oligonucleotide primer PCR (DOP-PCR), in which random primers that also contain universal amplification sequences are used in a first round of annealing and PCR. Then, a second round of PCR is used to amplify the sequences further with the universal primer sequences. In another embodiment, WGA is performed using multiple displacement amplification (MDA), which uses the phi-29 polymerase, which is a highly processive and non-specific enzyme that replicates nucleic acids and has been used for single-cell analysis. In some embodiments, WGA is not performed.

In some embodiments, selective amplification or enrichment are used to amplify or enrich target loci. In some embodiments, the amplification and/or selective enrichment technique may involve PCR such as ligation mediated PCR, fragment capture by hybridization, Molecular Inversion Probes, or other circularizing probes. In some embodiments, real-time quantitative PCR (RT-qPCR), digital PCR, droplet PCR, or emulsion PCR, single allele base extension reaction followed by mass spectrometry are used (Hung et al., J Clin Pathol 62:308-313, 2009, which is hereby incorporated by reference in its entirety). In some embodiments, capture by hybridization with hybrid capture probes is used to preferentially enrich the nucleic acids. In some embodiments, methods for amplification or selective enrichment may involve using probes where, upon correct hybridization to the target sequence, the 3-prime end or 5-prime end of a nucleotide probe is separated from the polymorphic site of a polymorphic allele by a small number of nucleotides. This separation reduces preferential amplification of one allele, termed allele bias. This is an improvement over methods that involve using probes where the 3-prime end or 5-prime end of a correctly hybridized probe are directly adjacent to or very near to the polymorphic site of an allele. In an embodiment, probes in which the hybridizing region may or certainly contains a polymorphic site are excluded. Polymorphic sites at the site of hybridization can cause unequal hybridization or inhibit hybridization altogether in some alleles, resulting in preferential amplification of certain alleles. These embodiments are improvements over other methods that involve targeted amplification and/or selective enrichment in that they better preserve the original allele frequencies of the sample at each polymorphic locus, whether the sample is pure genomic sample from a single individual or mixture of individuals

In some embodiments, a PCR technique referred to as mini-PCR is used to generate very short amplicons (U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, filed Nov. 18, 2011, and U.S. Ser. No. 61/994,791, filed May 16, 2014, which are each hereby incorporated by reference in its entirety). cfDNA is highly fragmented. For some cfDNA, the fragment sizes are distributed in approximately a Gaussian fashion with a mean of 160 bp, a standard deviation of 15 bp, a minimum size of about 100 bp, and a maximum size of about 220 bp. The polymorphic site of one particular target locus may occupy any position from the start to the end among the various fragments originating from that locus. Because cfDNA fragments are short, the likelihood of both primer sites being present the likelihood of a fragment of length L comprising both the forward and reverse primers sites is the ratio of the length of the amplicon to the length of the fragment. Under ideal conditions, assays in which the amplicon is 45, 50, 55, 60, 65, or 70 bp will successfully amplify from 72%, 69%, 66%, 63%, 59%, or 56%, respectively, of available template fragment molecules. In certain implementations, the cfDNA is amplified using primers that yield a maximum amplicon length of 85, 80, 75 or 70 bp, and in certain preferred embodiments 75 bp, and that have a melting temperature between 50 and 65° C., and in certain preferred embodiments, between 54-60.5° C. The amplicon length is the distance between the 5-prime ends of the forward and reverse priming sites. Amplicon length that is shorter than typically used may result in more efficient measurements of the desired polymorphic loci by only requiring short sequence reads. In an embodiment, a substantial fraction of the amplicons are less than 100 bp, less than 90 bp, less than 80 bp, less than 70 bp, less than 65 bp, less than 60 bp, less than 55 bp, less than 50 bp, or less than 45 bp.

In some embodiments, amplification is performed using direct multiplexed PCR, sequential PCR, nested PCR, doubly nested PCR, one-and-a-half sided nested PCR, fully nested PCR, one sided fully nested PCR, one-sided nested PCR, hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, which are hereby incorporated by reference in their entireties. If desired, any of these methods can be used for mini-PCR.

If desired, the extension step of the PCR amplification may be limited from a time standpoint to reduce amplification from fragments longer than 200 nucleotides, 300 nucleotides, 400 nucleotides, 500 nucleotides or 1,000 nucleotides. This may result in the enrichment of fragmented or shorter nucleic acids (such as cfDNA from cells that have undergone apoptosis or necrosis) and improvement of test performance.

In some embodiments, multiplex PCR is used. In some embodiments, the method of amplifying target loci in a nucleic acid sample involves (i) contacting the nucleic acid sample with a library of primers that simultaneously hybridize to least 100; 200; 500; 750; 1,000; 2,000; 5,000; 7,500; 10,000; 20,000; 25,000; 30,000; 40,000; 50,000; 75,000; or 100,000 different target loci to produce a reaction mixture; and (ii) subjecting the reaction mixture to primer extension reaction conditions (such as PCR conditions) to produce amplified products that include target amplicons. In some embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some embodiments, the primers are in solution (such as being dissolved in the liquid phase rather than in a solid phase). In some embodiments, the primers are in solution and are not immobilized on a solid support. In some embodiments, the primers are not part of a microarray. In some embodiments, the primers do not include molecular inversion probes (MIPs).

In some embodiments, two or more (such as 3 or 4) target amplicons (such as amplicons from the miniPCR method disclosed herein) are ligated together and then the ligated products are sequenced. Combining multiple amplicons into a single ligation product increases the efficiency of the subsequent sequencing step. In some embodiments, the target amplicons are less than 150, 100, 90, 75, or 50 base pairs in length before they are ligated. The selective enrichment and/or amplification may involve tagging each individual molecule with different tags, molecular barcodes, tags for amplification, and/or tags for sequencing. In some embodiments, the amplified products are analyzed by sequencing (such as by high throughput sequencing) or by hybridization to an array, such as a SNP array, the ILLUMINA INFINIUM array, or the AFFYMETRIX gene chip. In some embodiments, nanopore sequencing is used, such as the nanopore sequencing technology developed by Genia (see, for example, the world wide web at geniachip.com/technology, which is hereby incorporated by reference in its entirety). In some embodiments, duplex sequencing is used (Schmitt et al., “Detection of ultra-rare mutations by next-generation sequencing,” Proc Natl Acad Sci USA. 109(36): 14508-14513, 2012, which is hereby incorporated by reference in its entirety). This approach greatly reduces errors by independently tagging and sequencing each of the two strands of a DNA duplex. As the two strands are complementary, true mutations are found at the same position in both strands. In contrast, PCR or sequencing errors result in mutations in only one strand and can thus be discounted as technical error. In some embodiments, the method entails tagging both strands of duplex DNA with a random, yet complementary double-stranded nucleotide sequence, referred to as a Duplex Tag. Double-stranded tag sequences are incorporated into sequencing adapters by first introducing a single-stranded randomized nucleotide sequence into one adapter strand and then extending the opposite strand with a DNA polymerase to yield a complementary, double-stranded tag. Following ligation of tagged adapters to sheared DNA, the individually labeled strands are PCR amplified from asymmetric primer sites on the adapter tails and subjected to paired-end sequencing. In some embodiments, a sample (such as a DNA sample) or a preparation of nucleic acids is divided into multiple fractions, such as different wells (e.g., wells of a WaferGen SmartChip). Dividing the sample or preparation into different fractions (such as at least 5, 10, 20, 50, 75, 100, 150, 200, or 300 fractions) can increase the sensitivity of the analysis since the percent of molecules with a mutation are higher in some of the wells than in the overall sample. In some embodiments, each fraction has fewer than 500, 400, 200, 100, 50, 20, 10, 5, 2, or 1 nucleic acid molecule(s). In some embodiments, the molecules in each fraction are sequenced separately. In some embodiments, the same barcode (such as a random or non-human sequence) is added to all the molecules in the same fraction (such as by amplification with a primer containing the barcode or by ligation of a barcode), and different barcodes are added to molecules in different fractions. The barcoded molecules can be pooled and sequenced together. In some embodiments, the molecules are amplified before they are pooled and sequenced, such as by using nested PCR. In some embodiments, one forward and two reverse primers, or two forward and one reverse primers are used.

In some embodiments, a mutation (such as an SNV or CNV) that is present in less than 10, 5, 2, 1, 0.5, 0.1, 0.05, 0.01, or 0.005% of the DNA molecules in a sample (such as a sample of cfDNA) or a preparation of nucleic acids is measured (or is capable of being measured). In some embodiments, a mutation (such as an SNV or CNV) that is present in fewer than 1,000, 500, 100, 50, 20, 10, 5, 4, 3, or 2 original nucleic acid molecules (before amplification) in a sample (such as a sample of cfDNA from, e.g., a blood sample, a perfusate sample, or a flush sample) or a preparation of nucleic acids is measured (or is capable of being measured). In some embodiments, a mutation (such as an SNV or CNV) that is present in only 1 original nucleic acid molecule (before amplification) in a sample (such as a sample of cfDNA from, e.g., a blood sample, a perfusate sample, or a flush sample) or a preparation of nucleic acids is measured (or is capable of being measured).

For example, if the limit of detection of a mutation (such as a single nucleotide variant (SNV)) is 0.1%, a mutation present at 0.01% can be measured by dividing the fraction into multiple, fractions such as 100 wells. Most of the wells have no copies of the mutation. For the few wells with the mutation, the mutation is at a much higher percentage of the reads. In one example, there are 20,000 initial copies of DNA from the target locus, and two of those copies include a SNV of interest. If the sample or preparation is divided into 100 wells, 98 wells have the SNV, and 2 wells have the SNV at 0.5%. The nucleic acids in each well can be barcoded, amplified, pooled with nucleic acids from the other wells, and sequenced. Wells without the SNV can be used to measure the background amplification/sequencing error rate to evaluate if the signal from the outlier wells is above the background level of noise.

In some embodiments, the amplified products are measured using an array, such as an array especially a microarray with probes to one or more chromosomes of interest (e.g., chromosome 13, 18, 21, X, Y, or any combination thereof). It will be understood for example, that a SNP detection microarray could be used such as, for example, the Illumina (San Diego, Calif.) GoldenGate, DASL, Infinium, or CytoSNP-12 genotyping assay, or a SNP detection microarray product from Affymetrix, such as the OncoScan microarray. In some embodiments, phased genetic data for one or both biological parents of the embryo or fetus is used to increase the accuracy of analysis of array data from a single cell.

In some embodiments involving sequencing, the depth of read is the number of sequencing reads that map to a given locus. The depth of read may be normalized over the total number of reads. In some embodiments for depth of read of a sample or preparation, the depth of read is the average depth of read over the targeted loci. In some embodiments for the depth of read of a locus, the depth of read is the number of reads measured by the sequencer mapping to that locus. In general, the greater the depth of read of a locus, the closer the ratio of alleles at the locus tend to be to the ratio of alleles in the original sample or preparation of nucleic acids. Depth of read can be expressed in variety of different ways, including but not limited to the percentage or proportion. Thus, for example in a highly parallel DNA sequencer such as an Illumina HISEQ, which, e.g., produces a sequence of 1 million clones, the sequencing of one locus 3,000 times results in a depth of read of 3,000 reads at that locus. The proportion of reads at that locus is 3,000 divided by 1 million total reads, or 0.3% of the total reads.

In some embodiments, allelic data is obtained, wherein the allelic data includes quantitative measurement(s) indicative of the number of copies of a specific allele of a polymorphic locus. In some embodiments, the allelic data includes quantitative measurement(s) indicative of the number of copies of each of the alleles observed at a polymorphic locus. Quantitative measurements may be obtained for all possible alleles of the polymorphic locus of interest. For example, any of the methods discussed in the preceding paragraphs for evaluating the allele for a SNP or SNV locus, such as for example, microarrays, qPCR, RNA sequencing, DNA sequencing, such as high throughput DNA sequencing, can be used to generate quantitative measurements of the number of copies of a specific allele of a polymorphic locus. This quantitative measurement is referred to herein as allelic frequency data or measured genetic allelic data. Methods using allelic data are sometimes referred to as quantitative allelic methods; this is in contrast to quantitative methods which exclusively use quantitative data from non-polymorphic loci, or from polymorphic loci but without regard to allelic identity. When the allelic data is measured using high-throughput sequencing, the allelic data may include the number of reads of each allele mapping to the locus of interest.

In some embodiments, non-allelic data is obtained, wherein the non-allelic data includes quantitative measurement(s) indicative of the number of copies of a specific locus. The locus may be polymorphic or non-polymorphic. In some embodiments when the locus is non-polymorphic, the non-allelic data does not contain information about the relative or absolute quantity of the individual alleles that may be present at that locus. Methods using non-allelic data only (that is, quantitative data from non-polymorphic alleles, or quantitative data from polymorphic loci but without regard to the allelic identity of each fragment) are referred to as quantitative methods. Quantitative measurements may be obtained for all possible alleles of the polymorphic locus of interest, with one value associated with the measured quantity for all of the alleles at that locus, in total. Non-allelic data for a polymorphic locus may be obtained by summing the quantitative allelic for each allele at that locus. When the allelic data is measured using high-throughput sequencing, the non-allelic data may include the number of reads of mapping to the locus of interest. The sequencing measurements could indicate the relative and/or absolute number of each of the alleles present at the locus, and the non-allelic data includes the sum of the reads, regardless of the allelic identity, mapping to the locus. In some embodiments the same set of sequencing measurements can be used to yield both allelic data and non-allelic data. In some embodiments, the allelic data is used as part of a method to evaluate copy number at a chromosome of interest, and the produced non-allelic data can be used as part of a different method to evaluate copy number at a chromosome of interest. In some embodiments, the two methods are statistically orthogonal, and are combined to give a more accurate evaluation of the copy number at the chromosome of interest.

In some embodiments obtaining genetic data includes (i) acquiring nucleic acid sequence information by laboratory techniques, e.g., by the use of an automated high throughput sequencer, or (ii) acquiring information that had been previously obtained by laboratory techniques, wherein the information is electronically transmitted, e.g., by a computer over the internet or by electronic transfer from the sequencing device.

Additional exemplary sample preparation, amplification, and quantification methods are described in U.S. application Ser. No. 13/683,604, filed Nov. 21, 2012 (U.S. Publication No. 2013/0123120 and U.S. Ser. No. 61/994,791, filed May 16, 2014, which is hereby incorporated by reference in its entirety). These methods can be used for analysis of any of the samples or preparations disclosed herein.

Improved PCR amplification methods have also been developed that minimize or prevent interference due to the amplification of nearby or adjacent target loci in the same reaction volume (such as part of the sample multiplex PCR reaction that simultaneously amplifies all the target loci). These methods can be used to simultaneously amplify nearby or adjacent target loci, which is faster and cheaper than having to separate nearby target loci into different reaction volumes so that they can be amplified separately to avoid interference.

In some embodiments, the amplification of target loci is performed using a polymerase (e.g., a DNA polymerase, or reverse transcriptase) with low 5′-3′ exonuclease and/or low strand displacement activity. In some embodiments, the low level of 5′-3′ exonuclease reduces or prevents the degradation of a nearby primer (e.g., an unextended primer or a primer that has had one or more nucleotides added to during primer extension). In some embodiments, the low level of strand displacement activity reduces or prevents the displacement of a nearby primer (e.g., an unextended primer or a primer that has had one or more nucleotides added to it during primer extension). In some embodiments, target loci that are adjacent to each other (e.g., no bases between the target loci) or nearby (e.g., loci are within 50, 40, 30, 20, 15, 10, 9, 8, 7, 6, 5, 4, 3, 2, or 1 base) are amplified. In some embodiments, the 3′ end of one locus is within 50, 40, 30, 20, 15, 10, 9, 8, 7, 6, 5, 4, 3, 2, or 1 base of the 5′ end of next downstream locus.

In some embodiments, at least 100, 200, 500, 750, 1,000; 2,000; 5,000; 7,500; 10.000; 20,000; 25,000; 30,000; 40,000; 50,000; 75,000; or 100,000 different target loci are amplified, such as by the simultaneous amplification in one reaction volume. In some embodiments, at least 50, 60, 70, 80, 90, 95, 96, 97, 98, 99, or 99.5% of the amplified products are target amplicons. In various embodiments, the amount of amplified products that are target amplicons is between 50 to 99.5%, such as between 60 to 99%, 70 to 98%, 80 to 98%, 90 to 99.5%, or 95 to 99.5%, inclusive. In some embodiments, at least 50, 60, 70, 80, 90, 95, 96, 97, 98, 99, or 99.5% of the targeted loci are amplified (e.g., amplified at least 5, 10, 20, 30, 50, or 100-fold compared to the amount prior to amplification), such as by the simultaneous amplification in one reaction volume. In various embodiments, the amount target loci that are amplified (e.g., amplified at least 5, 10, 20, 30, 50, or 100-fold compared to the amount prior to amplification) is between 50 to 99.5%, such as between 60 to 99%, 70 to 98%, 80 to 99%, 90 to 99.5%, 95 to 99.9%, or 98 to 99.99% inclusive. In some embodiments, fewer non-target amplicons are produced, such as fewer amplicons formed from a forward primer from a first primer pair and a reverse primer from a second primer pair. Such undesired non-target amplicons can be produced using prior amplification methods if, e.g., the reverse primer from the first primer pair and/or the forward primer from the second primer pair are degraded and/or displaced.

In some embodiments, these methods allows longer extension times to be used since the polymerase bound to a primer being extended is less likely to degrade and/or displace a nearby primer (such as the next downstream primer) given the low 5′-3′ exonuclease and/or low strand displacement activity of the polymerase. In various embodiments, reaction conditions (such as the extension time and temperature) are used such that the extension rate of the polymerase allows the number of nucleotides that are added to a primer being extended to be equal to or greater than 80, 90, 95, 100, 110, 120, 130, 140, 150, 175, or 200% of the number of nucleotides between the 3′ end of the primer binding site and the 5′ end of the next downstream primer binding site on the same strand.

In some embodiments, a DNA polymerase is used produce DNA amplicons using DNA as a template. In some embodiments, a RNA polymerase is used produce RNA amplicons using DNA as a template. In some embodiments, a reverse transcriptase is used produce cDNA amplicons using RNA as a template.

In some embodiments, the low level of 5′-3′ exonuclease of the polymerase is less than 80, 70, 60, 50, 40, 30, 20, 10, 5, 1, or 0.1% of the activity of the same amount of Thermus aquaticus polymerase (“Taq” polymerase, which is a commonly used DNA polymerase from a thermophilic bacterium, PDB 1BGX, EC 2.7.7.7, Murali et al., “Crystal structure of Taq DNA polymerase in complex with an inhibitory Fab: the Fab is directed against an intermediate in the helix-coil dynamics of the enzyme,” Proc. Natl. Acad. Sci. USA 95:12562-12567, 1998, which is hereby incorporated by reference in its entirety) under the same conditions. In some embodiments, the low level of strand displacement activity of the polymerase is less than 80, 70, 60, 50, 40, 30, 20, 10, 5, 1, or 0.1% of the activity of the same amount of Taq polymerase under the same conditions.

In some embodiments, the polymerase is a PUSHION DNA polymerase, such as PHUSION High Fidelity DNA polymerase (M0530S, New England BioLabs, Inc.) or PHUSION Hot Start Flex DNA polymerase (M0535S, New England BioLabs, Inc.; Frey and Suppman BioChemica. 2:34-35, 1995; Chester and Marshak Analytical Biochemistry. 209:284-290, 1993, which are each hereby incorporated by reference in its entirety). The PHUSION DNA polymerase is a Pyrococcus-like enzyme fused with a processivity-enhancing domain. PHUSION DNA polymerase possesses 5′→3′ polymerase activity and 3′→5′ exonuclease activity, and generates blunt-ended products. PHUSION DNA polymerase lacks 5′→3′ exonuclease activity and strand displacement activity.

In some embodiments, the polymerase is a Q5® DNA Polymerase, such as Q5® High-Fidelity DNA Polymerase (M0491S, New England BioLabs, Inc.) or Q5® Hot Start High-Fidelity DNA Polymerase (M0493S, New England BioLabs, Inc.). Q5® High-Fidelity DNA polymerase is a high-fidelity, thermostable, DNA polymerase with 3′→5′ exonuclease activity, fused to a processivity-enhancing Sso7d domain. Q5® High-Fidelity DNA polymerase lacks 5′-3′ exonuclease activity and strand displacement activity.

In some embodiments, the polymerase is a T4 DNA polymerase (M0203S, New England BioLabs, Inc.; Tabor and Struh. (1989). “DNA-Dependent DNA Polymerases,” In Ausebel et al. (Ed.), Current Protocols in Molecular Biology. 3.5.10-3.5.12. New York: John Wiley & Sons, Inc., 1989; Sambrook et al. Molecular Cloning: A Laboratory Manual. (2nd ed.), 5.44-5.47. Cold Spring Harbor: Cold Spring Harbor Laboratory Press, 1989, which are each hereby incorporated by reference in its entirety). T4 DNA Polymerase catalyzes the synthesis of DNA in the 5′-3′ direction and requires the presence of template and primer. This enzyme has a 3′-5′ exonuclease activity which is much more active than that found in DNA Polymerase I. T4 DNA polymerase lacks 5′-3′ exonuclease activity and strand displacement activity.

In some embodiments, the polymerase is a Sulfolobus DNA Polymerase IV (M0327S, New England BioLabs, Inc.; (Boudsocq, et al. (2001). Nucleic Acids Res., 29:4607-4616, 2001; McDonald, et al. (2006). Nucleic Acids Res., 34:1102-1111, 2006, which are each hereby incorporated by reference in its entirety). Sulfolobus DNA Polymerase IV is a thermostable Y-family lesion-bypass DNA Polymerase that efficiently synthesizes DNA across a variety of DNA template lesions McDonald, J. P. et al. (2006). Nucleic Acids Res., 34, 1102-1111, which is hereby incorporated by reference in its entirety). Sulfolobus DNA Polymerase IV lacks 5′→3′ exonuclease activity and strand displacement activity.

In some embodiments, if a primer binds a region with a SNP, the primer may bind and amplify the different alleles with different efficiencies or may only bind and amplify one allele. For subjects who are heterozygous, one of the alleles may not be amplified by the primer. In some embodiments, a primer is designed for each allele. For example, if there are two alleles (e.g., a biallelic SNP), then two primers can be used to bind the same location of a target locus (e.g., a forward primer to bind the “A” allele and a forward primer to bind the “B” allele). Methods, such as, but not limited to, the dbSNP database, can be used to evaluate the location of known SNPs, such as SNP hot spots that have a high heterozygosity rate.

In some embodiments, the amplicons are similar in size. In some embodiments, the range of the length of the target amplicons is less than 100, 75, 50, 25, 15, 10, or 5 nucleotides. In some embodiments (such as the amplification of target loci in fragmented nucleic acids), the length of the target amplicons is between 50 and 100 nucleotides, such as between 60 and 80 nucleotides, or 60 and 75 nucleotides, inclusive. In some embodiments (such as the amplification of multiple target loci throughout an exon or gene), the length of the target amplicons is between 100 and 500 nucleotides, such as between 150 and 450 nucleotides, 200 and 400 nucleotides, 200 and 300 nucleotides, or 300 and 400 nucleotides, inclusive.

In some embodiments, multiple target loci are simultaneously amplified using a primer pair that includes a forward and reverse primer for each target locus to be amplified in that reaction volume. In some embodiments, one round of PCR is performed with a single primer per target locus, and then a second round of PCR is performed with a primer pair per target locus. For example, the first round of PCR may be performed with a single primer per target locus such that all the primers bind the same strand (such as using a forward primer for each target locus). This allows the PCR to amplify in a linear manner and reduces or eliminates amplification bias between amplicons due to sequence or length differences. In some embodiments, the amplicons are then amplified using a forward and reverse primer for each target locus.

If desired, multiplex PCR may be performed using primers with a decreased likelihood of forming primer dimers. In particular, highly multiplexed PCR can often result in the production of a very high proportion of product nucleic acids that results from unproductive side reactions such as primer dimer formation. In an embodiment, the particular primers that are most likely to cause unproductive side reactions may be removed from the primer library to give a primer library that will result in a greater proportion of amplified nucleic acids that map to the genome. The step of removing problematic primers, that is, those primers that are particularly likely to firm dimers has unexpectedly enabled extremely high PCR multiplexing levels for subsequent analysis by sequencing.

There are a number of ways to choose primers for a library where the amount of non-mapping primer dimer or other primer mischief products are minimized. Empirical data indicate that a small number of ‘bad’ primers are responsible for a large amount of non-mapping primer dimer side reactions. Removing these ‘bad’ primers can increase the percent of sequence reads that map to targeted loci. One way to identify the ‘bad’ primers is to look at the sequencing data of nucleic acids that were amplified by targeted amplification; those primer dimers that are seen with greatest frequency can be removed to give a primer library that is significantly less likely to result in side product nucleic acids that do not map to the genome. There are also programs that can calculate the binding energy of various primer combinations, and removing those with the highest binding energy will also give a primer library that is significantly less likely to result in side product nucleic acids that do not map to the genome.

In some embodiments for selecting primers, an initial library of candidate primers is created by designing one or more primers or primer pairs to candidate target loci. A set of candidate target loci (such as SNPs) can selected based on information about desired parameters for the target loci, such as frequency of the SNPs within a target population or the heterozygosity rate of the SNPs. In one embodiment, the PCR primers may be designed using the Primer3 program (the worldwide web at primer3.sourceforge.net; libprimer3 release 2.2.3, which is hereby incorporated by reference in its entirety). If desired, the primers can be designed to anneal within a particular annealing temperature range, have a particular range of GC contents, have a particular size range, produce target amplicons in a particular size range, and/or have other parameter characteristics. Starting with multiple primers or primer pairs per candidate target locus increases the likelihood that a primer or prime pair will remain in the library for most or all of the target loci. In one embodiment, the selection criteria may require that at least one primer pair per target locus remains in the library. That way, most or all of the target loci will be amplified when using the final primer library. This is desirable for applications such as screening for deletions or duplications at a large number of locations in the genome or screening for a large number of sequences (such as polymorphisms or other mutations) associated with a disease or an increased risk for a disease. If a primer pair from the library would produces a target amplicon that overlaps with a target amplicon produced by another primer pair, one of the primer pairs may be removed from the library to prevent interference.

In some embodiments, an “undesirability score” (higher score representing least desirability) is calculated (such as calculation on a computer) for most or all of the possible combinations of two primers from a library of candidate primers. In various embodiments, an undesirability score is calculated for at least 80, 90, 95, 98, 99, or 99.5% of the possible combinations of candidate primers in the library. Each undesirability score is based at least in part on the likelihood of dimer formation between the two candidate primers. If desired, the undesirability score may also be based on one or more other parameters selected from the group consisting of heterozygosity rate of the target locus, disease prevalence associated with a sequence (e.g., a polymorphism) at the target locus, disease penetrance associated with a sequence (e.g., a polymorphism) at the target locus, specificity of the candidate primer for the target locus, size of the candidate primer, melting temperature of the target amplicon, GC content of the target amplicon, amplification efficiency of the target amplicon, size of the target amplicon, and distance from the center of a recombination hotspot. In some embodiments, the specificity of the candidate primer for the target locus includes the likelihood that the candidate primer will mis-prime by binding and amplifying a locus other than the target locus it was designed to amplify. In some embodiments, one or more or all the candidate primers that mis-prime are removed from the library. In some embodiments to increase the number of candidate primers to choose from, candidate primers that may mis-prime are not removed from the library. If multiple factors are considered, the undesirability score may be calculated based on a weighted average of the various parameters. The parameters may be assigned different weights based on their importance for the particular application that the primers will be used for. In some embodiments, the primer with the highest undesirability score is removed from the library. If the removed primer is a member of a primer pair that hybridizes to one target locus, then the other member of the primer pair may be removed from the library. The process of removing primers may be repeated as desired. In some embodiments, the selection method is performed until the undesirability scores for the candidate primer combinations remaining in the library are all equal to or below a minimum threshold. In some embodiments, the selection method is performed until the number of candidate primers remaining in the library is reduced to a desired number.

In various embodiments, after the undesirability scores are calculated, the candidate primer that is part of the greatest number of combinations of two candidate primers with an undesirability score above a first minimum threshold is removed from the library. This step ignores interactions equal to or below the first minimum threshold since these interactions are less significant. If the removed primer is a member of a primer pair that hybridizes to one target locus, then the other member of the primer pair may be removed from the library. The process of removing primers may be repeated as desired. In some embodiments, the selection method is performed until the undesirability scores for the candidate primer combinations remaining in the library are all equal to or below the first minimum threshold. If the number of candidate primers remaining in the library is higher than desired, the number of primers may be reduced by decreasing the first minimum threshold to a lower second minimum threshold and repeating the process of removing primers. If the number of candidate primers remaining in the library is lower than desired, the method can be continued by increasing the first minimum threshold to a higher second minimum threshold and repeating the process of removing primers using the original candidate primer library, thereby allowing more of the candidate primers to remain in the library. In some embodiments, the selection method is performed until the undesirability scores for the candidate primer combinations remaining in the library are all equal to or below the second minimum threshold, or until the number of candidate primers remaining in the library is reduced to a desired number.

If desired, primer pairs that produce a target amplicon that overlaps with a target amplicon produced by another primer pair can be divided into separate amplification reactions. Multiple PCR amplification reactions may be desirable for applications in which it is desirable to analyze all of the candidate target loci (instead of omitting candidate target loci from the analysis due to overlapping target amplicons).

These selection methods minimize the number of candidate primers that have to be removed from the library to achieve the desired reduction in primer dimers. By removing a smaller number of candidate primers from the library, more (or all) of the target loci can be amplified using the resulting primer library.

Multiplexing large numbers of primers imposes considerable constraint on the assays that can be included. Assays that unintentionally interact result in spurious amplification products. The size constraints of miniPCR may result in further constraints. In an embodiment, it is possible to begin with a very large number of potential SNP targets (between about 500 to greater than 1 million) and attempt to design primers to amplify each SNP. Where primers can be designed it is possible to attempt to identify primer pairs likely to form spurious products by evaluating the likelihood of spurious primer duplex formation between all possible pairs of primers using published thermodynamic parameters for duplex formation. Primer interactions may be ranked by a scoring function related to the interaction and primers with the worst interaction scores are eliminated until the number of primers desired is met. In cases where SNPs likely to be heterozygous are most useful, it is possible to also rank the list of assays and select the most heterozygous compatible assays. Experiments have validated that primers with high interaction scores are most likely to form primer dimers. At high multiplexing it is not possible to eliminate all spurious interactions, but it is essential to remove the primers or pairs of primers with the highest interaction scores in silico as they can dominate an entire reaction, greatly limiting amplification from intended targets. We have performed this procedure to create multiplex primer sets of up to and in some cases more than 10,000 primers. The improvement due to this procedure is substantial, enabling amplification of more than 80%, more than 90%, more than 95%, more than 98%, and even more than 99% on target products as measured by sequencing of all PCR products, as compared to 10% from a reaction in which the worst primers were not removed. When combined with a partial semi-nested approach as previously described, more than 90%, and even more than 95% of amplicons may map to the targeted sequences.

Note that there are other methods for evaluating which PCR probes are likely to form dimers. In an embodiment, analysis of a pool of nucleic acids that have been amplified using a non-optimized set of primers may be sufficient to identify problematic primers. For example, analysis may be done using sequencing, and those dimers which are present in the greatest number are identified to be those most likely to form dimers, and may be removed. In an embodiment, the method of primer design may be used in combination with the mini-PCR method described herein.

The use of tags on the primers may reduce amplification and sequencing of primer dimer products. In some embodiments, the primer contains an internal region that forms a loop structure with a tag. In particular embodiments, the primers include a 5′ region that is specific for a target locus, an internal region that is not specific for the target locus and forms a loop structure, and a 3′ region that is specific for the target locus. In some embodiments, the loop region may lie between two binding regions where the two binding regions are designed to bind to contiguous or neighboring regions of template nucleic acids. In various embodiments, the length of the 3′ region is at least 7 nucleotides. In some embodiments, the length of the 3′ region is between 7 and 20 nucleotides, such as between 7 to 15 nucleotides, or 7 to 10 nucleotides, inclusive. In various embodiments, the primers include a 5′ region that is not specific for a target locus (such as a tag or a universal primer binding site) followed by a region that is specific for a target locus, an internal region that is not specific for the target locus and forms a loop structure, and a 3′ region that is specific for the target locus. Tag-primers can be used to shorten necessary target-specific sequences to below 20, below 15, below 12, and even below 10 base pairs. This can be serendipitous with primer design when the target sequence is fragmented within the primer binding site or, or it can be designed into the primer design. Advantages of this method include: it increases the number of assays that can be designed for a certain maximal amplicon length, and it shortens the “non-informative” sequencing of primer sequence. It may also be used in combination with internal tagging.

In an embodiment, the relative amount of nonproductive products in the multiplexed targeted PCR amplification can be reduced by raising the annealing temperature. In cases where one is amplifying libraries with the same tag as the target specific primers, the annealing temperature can be increased in comparison to the genomic DNA as the tags will contribute to the primer binding. In some embodiments reduced primer concentrations are used, optionally along with longer annealing times. In some embodiments the annealing times may be longer than 3 minutes, longer than 5 minutes, longer than 8 minutes, longer than 10 minutes, longer than 15 minutes, longer than 20 minutes, longer than 30 minutes, longer than 60 minutes, longer than 120 minutes, longer than 240 minutes, longer than 480 minutes, and even longer than 960 minutes. In certain illustrative embodiments, longer annealing times are used along with reduced primer concentrations. In various embodiments, longer than normal extension times are used, such as greater than 3, 5, 8, 10, or 15 minutes. In some embodiments, the primer concentrations are as low as 50 nM, 20 nM, 10 nM, 5 nM, 1 nM, and lower than 1 nM. This surprisingly results in robust performance for highly multiplexed reactions, for example 1,000-plex reactions, 2,000-plex reactions, 5,000-plex reactions, 10,000-plex reactions, 20,000-plex reactions, 50,000-plex reactions, and even 100,000-plex reactions. In an embodiment, the amplification uses one, two, three, four or five cycles run with long annealing times, followed by PCR cycles with more usual annealing times with tagged primers.

To select target locations, one may start with a pool of candidate primer pair designs and create a thermodynamic model of potentially adverse interactions between primer pairs, and then use the model to eliminate designs that are incompatible with other the designs in the pool.

In an embodiment, the systems or methods feature a method of decreasing the number of target loci (such as loci that may contain a polymorphism or mutation associated with a disease or disorder or an increased risk for transplant rejection) and/or increasing the disease load that is measured (e.g., increasing the number of polymorphisms or mutations that are measured). In some embodiments, the method includes ranking (such as ranking from highest to lowest) loci by frequency or reoccurrence of a polymorphism or mutation (such as a single nucleotide variation, insertion, or deletion, or any of the other variations described herein) in each locus among subjects with the disease or disorder. In some embodiments, PCR primers are designed to some or all of the loci. During selection of PCR primers for a library of primers, primers to loci that have a higher frequency or reoccurrence (higher ranking loci) are favored over those with a lower frequency or reoccurrence (lower ranking loci). In some embodiments, this parameter is included as one of the parameters in the calculation of the undesirability scores described herein. If desired, primers (such as primers to high ranking loci) that are incompatible with other designs in the library can be included in a different PCR library/pool. In some embodiments, multiple libraries/pools (such as 2, 3, 4, 5 or more) are used in separate PCR reactions to enable amplification of all (or a majority) of the loci represented by all the libraries/pools. In some embodiment, this method is continued until sufficient primers are included in one or more libraries/pools such that the primers, in aggregate, enable the desired disease load to be captured for the disease or disorder (e.g., such as by measurement of at least 80, 85, 90, 95, or 99% of the disease load).

In some implementations, systems or methods described herein use libraries of primers, such as primers selected from a library of candidate primers using any of the methods described herein. In some embodiments, the library includes primers that simultaneously hybridize (or are capable of simultaneously hybridizing) to or that simultaneously amplify (or are capable of simultaneously amplifying) at least 100; 200; 500; 750; 1,000; 2,000; 5,000; 7,500; 10,000; 20,000; 25,000; 30,000; 40,000; 50,000; 75,000; or 100,000 different target loci in one reaction volume. In various embodiments, the library includes primers that simultaneously amplify (or are capable of simultaneously amplifying) between 100 to 500; 500 to 1,000; 1,000 to 2,000; 2,000 to 5,000; 5,000 to 7,500; 7,500 to 10,000; 10,000 to 20,000; 20,000 to 25,000; 25,000 to 30,000; 30,000 to 40,000; 40,000 to 50,000; 50,000 to 75,000; or 75,000 to 100,000 different target loci in one reaction volume, inclusive. In various embodiments, the library includes primers that simultaneously amplify (or are capable of simultaneously amplifying) between 1,000 to 100,000 different target loci in one reaction volume, such as between 1,000 to 50,000; 1,000 to 30,000; 1,000 to 20,000; 1,000 to 10,000; 2,000 to 30,000; 2,000 to 20,000; 2,000 to 10,000; 5,000 to 30,000; 5,000 to 20,000; or 5,000 to 10,000 different target loci, inclusive. In some embodiments, the library includes primers that simultaneously amplify (or are capable of simultaneously amplifying) the target loci in one reaction volume such that less than 60, 40, 30, 20, 10, 5, 4, 3, 2, 1, 0.5, 0.25, 0.1, or 0.5% of the amplified products are primer dimers. The various embodiments, the amount of amplified products that are primer dimers is between 0.5 to 60%, such as between 0.1 to 40%, 0.1 to 20%, 0.25 to 20%, 0.25 to 10%, 0.5 to 20%, 0.5 to 10%, 1 to 20%, or 1 to 10%, inclusive. In some embodiments, the primers simultaneously amplify (or are capable of simultaneously amplifying) the target loci in one reaction volume such that at least 50, 60, 70, 80, 90, 95, 96, 97, 98, 99, or 99.5% of the amplified products are target amplicons. In various embodiments, the amount of amplified products that are target amplicons is between 50 to 99.5%, such as between 60 to 99%, 70 to 98%, 80 to 98%, 90 to 99.5%, or 95 to 99.5%, inclusive. In some embodiments, the primers simultaneously amplify (or are capable of simultaneously amplifying) the target loci in one reaction volume such that at least 50, 60, 70, 80, 90, 95, 96, 97, 98, 99, or 99.5% of the targeted loci are amplified (e.g., amplified at least 5, 10, 20, 30, 50, or 100-fold compared to the amount prior to amplification). In various embodiments, the amount target loci that are amplified (e.g., amplified at least 5, 10, 20, 30, 50, or 100-fold compared to the amount prior to amplification) is between 50 to 99.5%, such as between 60 to 99%, 70 to 98%, 80 to 99%, 90 to 99.5%, 95 to 99.9%, or 98 to 99.99% inclusive. In some embodiments, the library of primers includes at least 100; 200; 500; 750; 1,000; 2,000; 5,000; 7,500; 10,000; 20,000; 25,000; 30,000; 40,000; 50,000; 75,000; or 100,000 primer pairs, wherein each pair of primers includes a forward test primer and a reverse test primer where each pair of test primers hybridize to a target locus. In some embodiments, the library of primers includes at least 100; 200; 500; 750; 1,000; 2,000; 5,000; 7,500; 10,000; 20,000; 25,000; 30,000; 40.000; 50,000; 75,000; or 100,000 individual primers that each hybridize to a different target locus, wherein the individual primers are not part of primer pairs.

In various embodiments, the concentration of each primer is less than 100, 75, 50, 25, 20, 10, 5, 2, or 1 nM, or less than 500, 100, 10, or 1 uM. In various embodiments, the concentration of each primer is between 1 uM to 100 nM, such as between 1 uM to 1 nM, 1 to 75 nM, 2 to 50 nM or 5 to 50 nM, inclusive. In various embodiments, the GC content of the primers is between 30 to 80%, such as between 40 to 70%, or 50 to 60%, inclusive. In some embodiments, the range of GC content of the primers is less than 30, 20, 10, or 5%. In some embodiments, the range of GC content of the primers is between 5 to 30%, such as 5 to 20% or 5 to 10%, inclusive. In some embodiments, the melting temperature (Tm) of the test primers is between 40 to 80° C., such as 50 to 70° C., 55 to 65° C., or 57 to 60.5° C., inclusive. In some embodiments, the Tm is calculated using the Primer3 program (libprimer3 release 2.2.3) using the built-in SantaLucia parameters (the world wide web at primer3.sourceforge.net). In some embodiments, the range of melting temperature of the primers is less than 15, 10, 5, 3, or 1° C. In some embodiments, the range of melting temperature of the primers is between 1 to 15° C., such as between 1 to 10° C., 1 to 5° C., or 1 to 3° C., inclusive. In some embodiments, the length of the primers is between 15 to 100 nucleotides, such as between 15 to 75 nucleotides, 15 to 40 nucleotides, 17 to 35 nucleotides, 18 to 30 nucleotides, or 20 to 65 nucleotides, inclusive. In some embodiments, the range of the length of the primers is less than 50, 40, 30, 20, 10, or 5 nucleotides. In some embodiments, the range of the length of the primers is between 5 to 50 nucleotides, such as 5 to 40 nucleotides, 5 to 20 nucleotides, or 5 to 10 nucleotides, inclusive. In some embodiments, the length of the target amplicons is between 50 and 100 nucleotides, such as between 60 and 80 nucleotides, or 60 to 75 nucleotides, inclusive. In some embodiments, the range of the length of the target amplicons is less than 50, 25, 15, 10, or 5 nucleotides. In some embodiments, the range of the length of the target amplicons is between 5 to 50 nucleotides, such as 5 to 25 nucleotides, 5 to 15 nucleotides, or 5 to 10 nucleotides, inclusive. In some embodiments, the library does not comprise a microarray. In some embodiments, the library comprises a microarray.

In some embodiments, some (such as at least 80, 90, or 95%) or all of the adaptors or primers include one or more linkages between adjacent nucleotides other than a naturally-occurring phosphodiester linkage. Examples of such linkages include phosphoramide, phosphorothioate, and phosphorodithioate linkages. In some embodiments, some (such as at least 80, 90, or 95%) or all of the adaptors or primers include a thiophosphate (such as a monothiophosphate) between the last 3′ nucleotide and the second to last 3′ nucleotide. In some embodiments, some (such as at least 80, 90, or 95%) or all of the adaptors or primers include a thiophosphate (such as a monothiophosphate) between the last 2, 3, 4, or 5 nucleotides at the 3′ end. In some embodiments, some (such as at least 80, 90, or 95%) or all of the adaptors or primers include a thiophosphate (such as a monothiophosphate) between at least 1, 2, 3, 4, or 5 nucleotides out of the last 10 nucleotides at the 3′ end. In some embodiments, such primers are less likely to be cleaved or degraded. In some embodiments, the primers do not contain an enzyme cleavage site (such as a protease cleavage site).

Additional exemplary multiplex PCR methods and libraries are described in U.S. application Ser. No. 13/683,604, filed Nov. 21, 2012 (U.S. Publication No. 2013/0123120) and U.S. Ser. No. 61/994,791, filed May 16, 2014, which are each hereby incorporated by reference in its entirety). These methods and libraries can be used for analysis of any of the samples or preparations disclosed herein and for use in any of the methods of the disclosure.

### Example #1

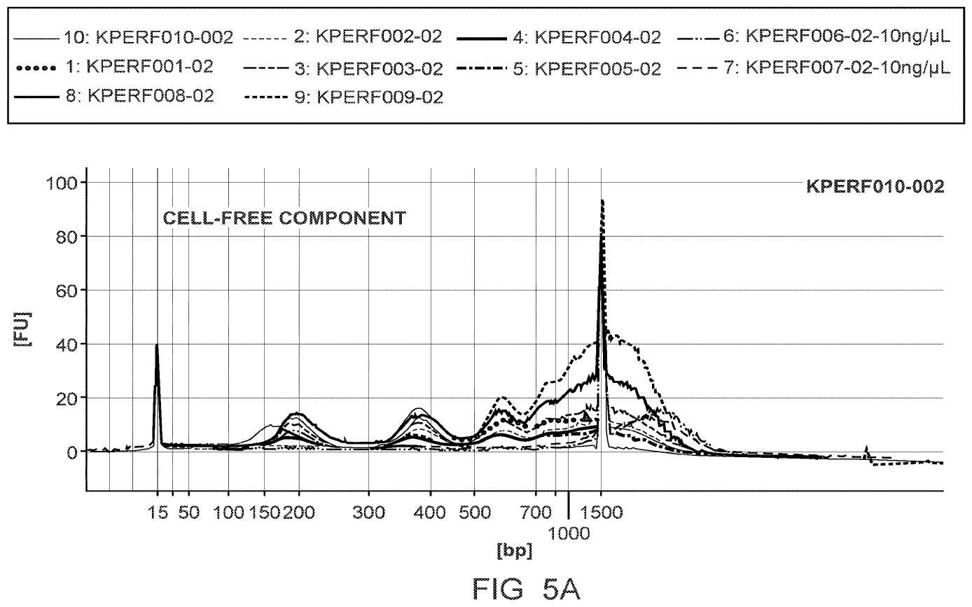

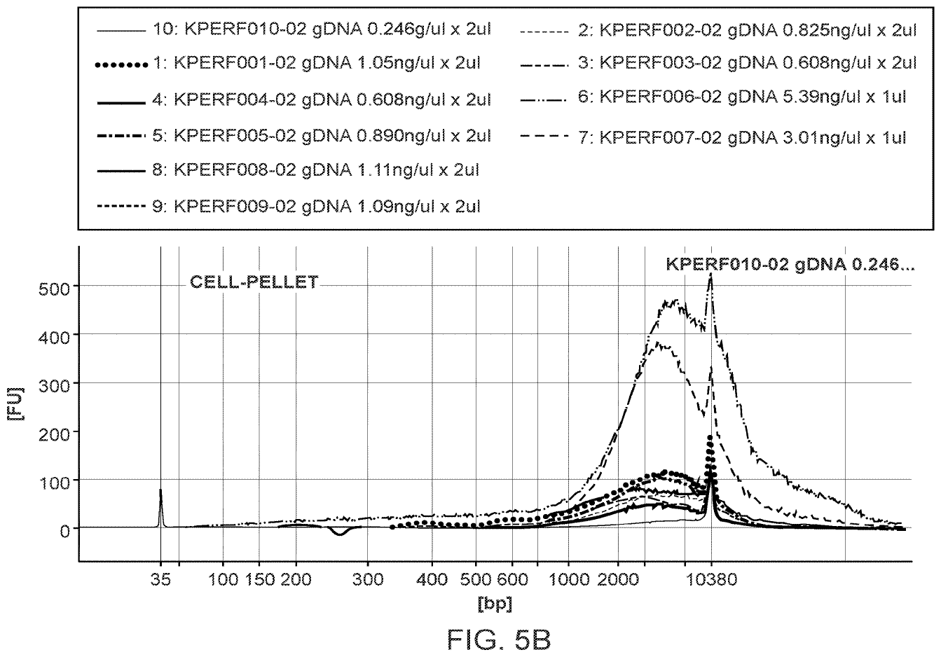

FIGS. 5A-9B depict the results of a study performed on perfusion fluid samples taken from donated kidneys. A cohort of seven donors donated kidneys, four of whom donated one kidney each and three of whom donated two kidneys each. The kidney samples are listed in Table 1. Four 10 mL aliquots of 10 perfusions for these kidneys were sampled. Each aliquot was centrifuged to separate a pellet of cells from the rest of the perfusion fluid. Cellular DNA was extracted from the cell pellets using the QIAAMP® DNA Micro Kit, and cfDNA was extracted from the perfusion fluid using a “NICE chemistry” protocol. Extracted DNA was quantified by the Invitrogen QUBIT® Fluorimeter (using the dsDNA BR Assay Kit) and fragment sizes were evaluated by the Agilent 2100 BIOANALYZER® system (using the High Sensitivity DNA assay and the DNA-1000 assay).

TABLE 1
Perfusion times and temperatures of
kidney samples used for the study.

| Subject ID | Kidney | Perfusion time (min) | Temp. ° C. |
|---|---|---|---|
| AHFK479 | — | — | — |
| AHFL447 | right | — | 1.7 |
| AHFL447 | left | — | 1.2 |
| AHFK342 | right | 744 | 2.0 |
| AHF2108 | left | 717 | 1.3 |
| AHF3305 | left | 1265 | 2.1 |
| AHF3305 | right | 1658 | 2.7 |
| AHGC414 | right | 1020 | 1.5 |
| AHGC414 | left | 1145 | 1.5 |
| AHGF262 | left | 660 | 2.7 |

FIGS. 5A and 5B show electropherograms of the cell-free and cellular components of DNA, respectively, from each of the kidney perfusion fluid samples. The horizontal axes represent fragment size in base pairs (bp), and the vertical axes represent relative levels of each fragment size based on fluorescence units (FU). In FIG. 5A, a lower marker (LM) is shown as a sharp peak at 15 bp, and an upper marker (UM) is shown as a sharp peak at 1500. In FIG. 5B, the LM is shown as a sharp peak at 35 bp, and the UM is shown as a sharp peak at 10,380 bp. The LM and UM are internal markers that are added during processing to ensure proper sizing of the samples and do not represent levels of sample DNA.

As seen in FIG. 5A, cfDNA is present in the perfusate samples in fragments of varying size, represented by the several small peaks at around 200 bp and 400 bp as well as higher molecular weights. This distribution of smaller cfDNA fragments (e.g., less than about 200 bp) may be indicative of DNA that was broken into fragments around nucleosomes and released from cells during apoptosis, because these peaks have the same height and are present at equal intervals across the spectrum corresponding to nucleosome size. In FIG. 5B, cellular DNA is present almost exclusively as high molecular weight fragments (e.g., greater than 200 bp).

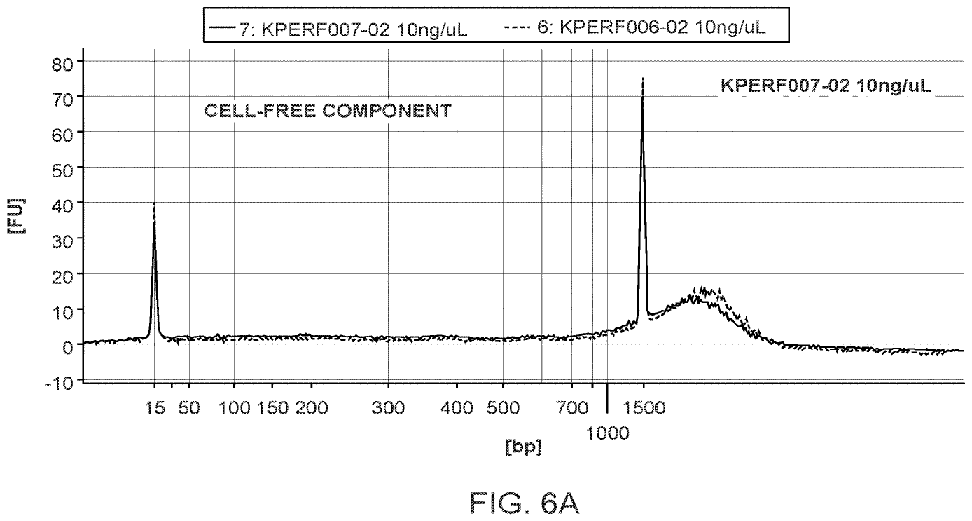

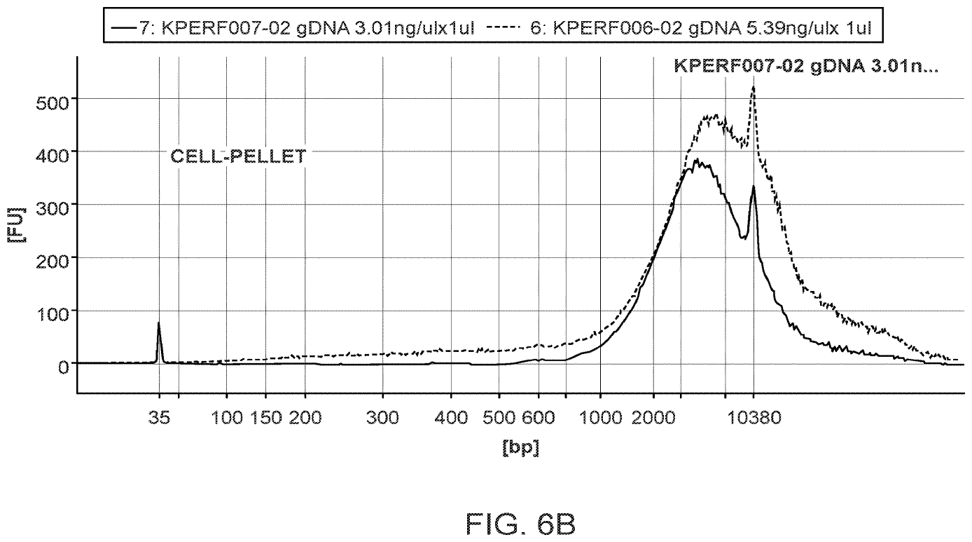

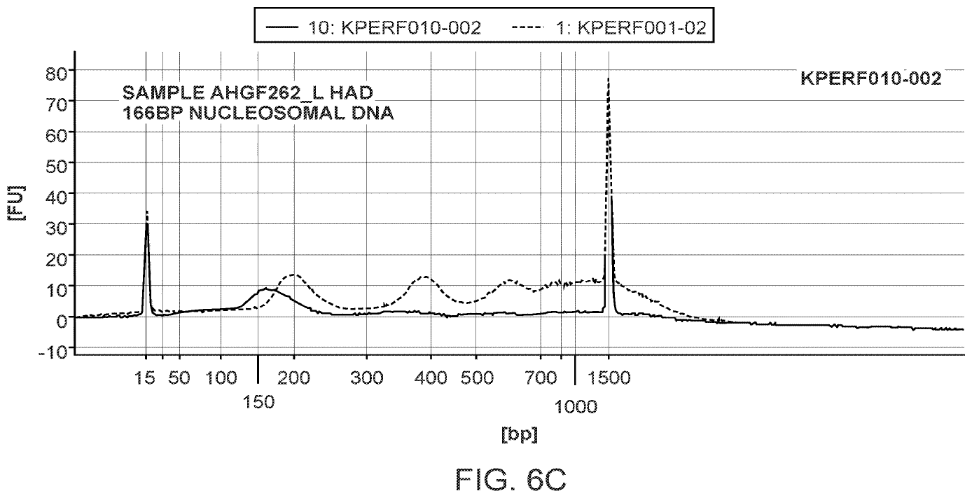

FIGS. 6A, 6B, and 6C show the electropherograms for particular samples having distinct size profiles. In FIG. 6A, the cfDNA size profile is shown for the sample which was evaluated to have the highest concentration of DNA among the full set of samples. This particular sample had only high molecular weight fragments at detectable levels, whereas any low molecular weight (nucleosomal) fragments were at undetectable levels. The corresponding cellular DNA profile is shown in FIG. 6B, where similarly only high molecular weight fragments are present at detectable levels. FIG. 6C shows the cfDNA electropherogram for a particular sample, AHGF262_L, which notably had a peak of 166 bp fragments, indicative of significant nucleosomal DNA presence.

Presence of nucleosomal peaks in samples may be used to evaluate the likely source of cfDNA, which may be correlated to donor organ/tissue health. For example, the sample of FIGS. 6A and 6B may be flagged for mechanical organ/tissue injury due to surgery or release of immune cells, but lack of nucleosomal fragmentation in the cfDNA (e.g., characteristic of cell necrosis), whereas the sample with fragment size of around 160-170 bp in FIG. 6C may be due to programmed cell death by apoptosis, which may persist after transplantation, while DNA in the other represented samples may have been a result of necrosis.

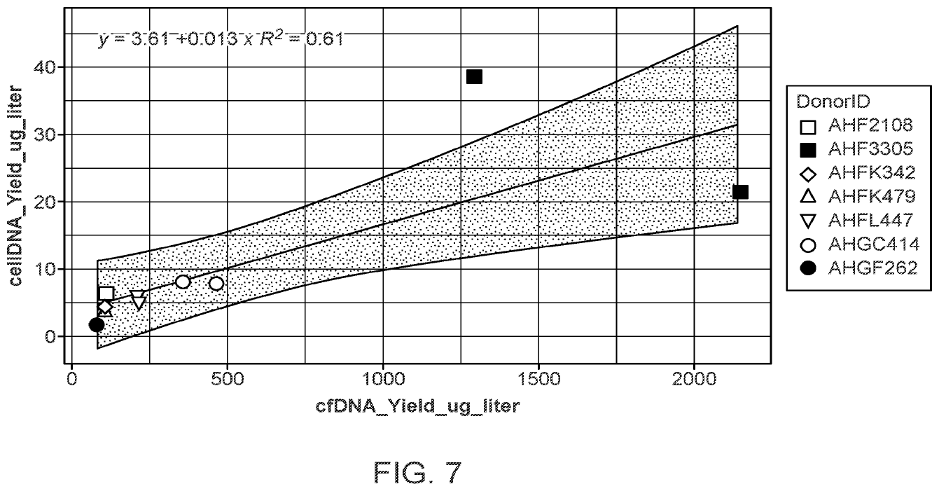

In FIG. 7, the yield of cellular DNA in g/L is plotted versus the yield of cfDNA in g/L for each of the samples. The amount of cellular DNA was only about 1.3% of the amount of cfDNA on average for each sample; however, there is a linear correlation between the relative yields of each fraction of DNA. Thus, one may be able to use quantity of cellular DNA or cfDNA to estimate the quantity of the other. The linear correlation has a slope of 0.013, y-intercept of 3.61, and a R-squared (coefficient of determination) value of 0.61.

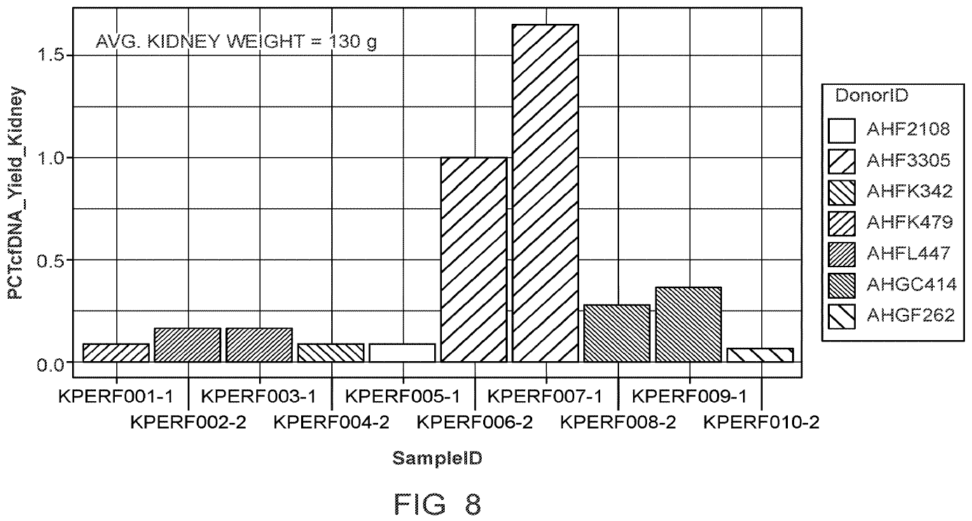

In FIG. 8, the percent cfDNA yield relative to kidney weight is charted for each sample. An average kidney weight of 130 g was reported. The two elevated bars are from the samples taken from the two kidneys of donor AHF3305, for which the electropherograms were shown in FIGS. 6A and 6B.

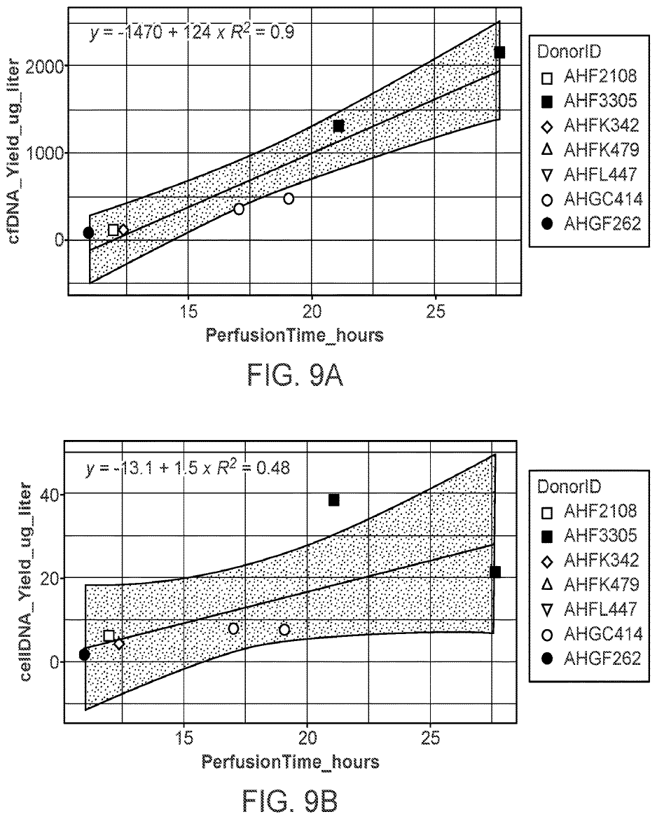

In FIGS. 9A and 9B, the yields of cfDNA and cellular DNA, respectively, in g/L are plotted against perfusion time (the length of time each kidney had been perfused up to taking the perfusate samples) in hours. In FIG. 9A, there is a strong linear correlation between cfDNA yield and perfusion time. The linear correlation has a slope of 124, a y-intercept of 1470, and a R-squared value of 0.9. In FIG. 9B, the linear correlation between cellular DNA yield and perfusion time has a slope of 1.5, a y-intercept of 13.1, and a R-squared value of 0.48. Notably, the samples having the longest perfusion time were the same samples showing the highest yields of cfDNA in FIG. 8, so extended perfusion may have led to injury of the kidney.

The foregoing is merely illustrative of the principles of the disclosure, and the apparatuses can be practiced by other than the described embodiments, which are presented for purposes of illustration and not of limitation. It is to be understood that the apparatuses disclosed herein, while shown for use in mechanical perfusion of a donor organ or tissue, may be applied to perfusion of other biological entities.

Variations and modifications will occur to those of skill in the art after reviewing this disclosure. The disclosed features may be implemented, in any combination and sub-combination (including multiple dependent combinations and sub-combinations), with one or more other features described herein. The various features described or illustrated above, including any components thereof, may be combined or integrated in other systems. Moreover, certain features may be omitted or not implemented.

The systems and methods described may be implemented by a physician or automatedly on a mechanical perfusion system and/or DNA analysis platform. The perfusion system and/or DNA analysis platform may include a data processing apparatus. The systems and methods described herein may be implemented remotely on a separate data processing apparatus. The separate data processing apparatus may be connected directly or indirectly to the system/platform through cloud applications. The system/platform may communicate with the separate data processing apparatus in real-time (or near real-time).

In general, embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter affecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program may correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices.

Examples of changes, substitutions, and alterations are ascertainable by one skilled in the art and could be made without departing from the scope of the information disclosed herein. All references cited herein are incorporated by reference in their entirety and made part of this application.

What is claimed is:

1. A method for preparing a non-naturally occurring nucleic acid sample from perfusion fluid or flush useful for assessing transplantation outcome of a donor organ or a donor tissue, the method comprising:
obtaining a sample of perfusion fluid or flush from a donor organ or a donor tissue that has been perfused with the perfusion fluid or prepared with the flush, the perfusion fluid or flush comprising nucleic acids;
isolating all nucleic acids from the sample; and
preparing the isolated nucleic acids to be assayed for measurement of at least one of an amount of nucleic acids in the perfusion fluid or flush, a molecular weight of nucleic acids in the perfusion fluid or flush, or a fragment size distribution of nucleic acids in the perfusion fluid or flush.

2. The method of claim 1, wherein the amount of nucleic acids is a total amount of DNA in the perfusion fluid or flush.

3. The method of claim 1, wherein the amount of nucleic acids is an amount of high molecular-weight DNA in the perfusion fluid or flush.

4. The method of claim 3, wherein the high molecular-weight DNA comprises DNA fragments having greater than 200 base pairs.

5. The method of claim 1, wherein the amount of nucleic acids is an amount of low molecular-weight DNA in the perfusion fluid or flush.

6. The method of claim 3, wherein the low molecular-weight DNA comprises DNA fragments having less than 200 base pairs

7. The method of claim 1, wherein the molecular weight of DNA is an average molecular weight of DNA in the perfusion fluid or flush.

8. The method of any of claims 1-7, wherein the isolated nucleic acids are cell-free DNA.

9. The method of claim 8, further comprising:
evaluating that the cell-free DNA is derived from apoptosis if the cell-free DNA has a molecular weight that is consistent with nucleosomal DNA.

10. The method of any of claims 1-7, wherein the isolated nucleic acids are cellular DNA.

11. The method of any of claims 1-7, wherein the isolated nucleic acids are RNA.

12. The method of claim 11, wherein the method further comprises:
evaluating a gene expression characteristic corresponding to the isolated RNA to evaluate a cellular origin of the RNA.

13. The method of claim 12, wherein the cellular origin of the RNA is evaluated to be an immune cell when the gene expression characteristic relates to immune function.

14. The method of any of claims 11-13, wherein isolating the RNA comprises lysing vesicles containing the RNA.

15. The method of any of claims 1-14, further comprising:
normalizing the amount of nucleic acids relative to a perfusion time.

16. The method of any of claims 1-15, further comprising:
normalizing the amount of nucleic acids relative to at least one of a size, weight, volume, or surface area of the donor organ or donor tissue.

17. The method of any of claims 1-16, further comprising:
normalizing the amount of DNA relative to a perfusion volume of the perfusion fluid or a flush volume of the flush.

18. The method of any of claims 1-17, further comprising:
normalizing the amount of nucleic acids relative to a temperature of the perfusion fluid or flush.

19. The method of any of claims 1-18, further comprising:
evaluating a fragmentation pattern of the nucleic acids.

20. The method of claim 19, further comprising:
based on the fragmentation pattern, categorizing the isolated nucleic acids into nucleic acids derived from random degradation of rupturing cells in the perfusion fluid or flush and nucleic acids derived from apoptosis of cells in the donor organ or donor tissue.

21. The method of any of claims 1-20, further comprising:
obtaining one or more additional samples of the perfusion fluid or flush taken from different points in time than the sample;
quantifying an amount of nucleic acids in the perfusion fluid or flush of the one or more additional samples; and
tracking the amount of nucleic acids in the perfusion fluid or flush over time based on the sample and the one or more additional samples.

22. The method of any of claims 1-21, wherein the measurement comprises at least one of electrophoresis, mass spectrometry, fluorimetry, qPCR, or droplet PCR.

23. The method of any of claims 1-22, comprising assaying the prepared isolated nucleic acids for measurement of at least one of an amount of nucleic acids in the perfusion fluid or flush, a molecular weight of nucleic acids in the perfusion fluid or flush, or a fragment size distribution of nucleic acids in the perfusion fluid or flush.

24. A method for assessing an outcome of a transplant, the method comprising:
preparing a nucleic acid preparation according to the method of any of claims 1-23; and
assessing an outcome of transplantation of the donor organ or donor tissue based on the amount of nucleic acids.

25. The method of claim 24, wherein the nucleic acids are cellular DNA from donor-derived cells, and wherein the predicted outcome is a predicted favorable or unfavorable prognosis based on the cellular DNA, the cellular DNA being indicative of an immune response.

26. The method of claim 24, wherein the nucleic acids are cell-free DNA, and wherein the predicted outcome is a predicted favorable or unfavorable prognosis based on the cell-free DNA, the cell-free DNA being indicative of damage to the donor organ or donor tissue.

27. The method of claim 24, wherein the predicted outcome is a predicted unfavorable prognosis being a decision maker for not using the organ for transplantation.

28. The method of any of claims 24-27, wherein the predicted outcome is predicted based at least in part on a risk call determined via an algorithm using the amount of nucleic acids as an algorithm input.

29. The method of any of claims 24-28, wherein the predicted outcome is predicted by performing a quality evaluation of the donor organ or donor tissue based on the amount of nucleic acids.

30. The method of claim 29, wherein the quality evaluation indicates at least one of the following characteristics associated with transplantation outcome: a presence of delayed graft function, a duration of delayed graft function, a rate of primary non-function, or an organ function at various time points after transplant.

31. A method for assessing an outcome of a transplant, the method comprising:
preparing a nucleic acid preparation according to the method of any of claims 1-23; and
assessing an outcome of the transplant based on at least a part of the fragment size distribution of the nucleic acids.

32. The method of claim 31, wherein the predicted outcome is predicted based at least in part on a risk call generated via an algorithm using the fragment size distribution or a subset of the fragment size distribution as an algorithm input.

33. The method of any of claims 24-32, wherein the predicted outcome is rejection of the transplantation or non-rejection of the transplant.

34. The method of any of claims 24-33, wherein the predicted outcome comprises a type of rejection of the transplant.

35. The method of any of claims 24-34, wherein the predicted outcome comprises a time of rejection of the transplant.

36. The method of any of claims 24-35, further comprising:
making a recommendation to cease perfusion, suspend transplantation, or delay transplantation of the organ or tissue based on the predicted outcome.

37. The method of claim 36, wherein the recommendation is made if the amount of nucleic acids exceeds a threshold amount of nucleic acids.

38. The method of any of claims 1-37, further comprising:
performing a targeted genetic analysis of the isolated nucleic acids to identify a genetic feature in the nucleic acids.

39. The method of claim 38, wherein the targeted genetic analysis comprises targeted amplification and high-throughput sequencing of at least 50 target loci in the nucleic acids.

40. The method of any of claims 38-39, further comprising:
evaluating a change in homeostasis or in cellular processes based on the targeted genetic analysis.

41. A method for preparing a non-naturally-occurring nucleic acid preparation from perfusion fluid or flush useful for assessing transplantation outcome of a donor organ or a donor tissue, the method comprising:
obtaining a sample of perfusion fluid or flush from a donor organ or a donor tissue that has been perfused with the perfusion fluid or prepared with the flush, the perfusion fluid or flush comprising nucleic acids;
isolating nucleic acids from the sample; and
preparing the isolated nucleic acids to be assayed for at least one of a type of nucleic acid in the sample or a sequence of the nucleic acids in the sample.

42. The method of claim 41, wherein the isolated nucleic acids are RNA.

43. The method of claim 42, wherein the type of nucleic acid is either a coding RNA or non-coding RNA.

44. The method of claim 43, further comprising:
if the type of nucleic acid is non-coding RNA, evaluating an expression modulation function of the isolated nucleic acids.

45. The method of claim 42, wherein the sequence is evaluated using high-throughput sequencing.

46. The method of any of claims 42-45, wherein isolating the RNA comprises lysing vesicles containing the RNA.

47. A method for feedback-controlled mechanical perfusion of a donor organ or donor tissue, comprising:
perfusing a donor organ or donor tissue at a first value of a perfusion parameter in a perfusion chamber holding the donor organ or donor tissue;
performing the method of any of claims 1-42 on the donor organ or donor tissue;
generating an appropriate adjustment to the perfusion parameter based at least in part on the measurement of the isolated nucleic acids; and
adjusting the perfusion parameter to a second value based on the generated appropriate adjustment.

48. The method of claim 47, wherein the adjustment is an increase or a decrease in a perfusion flow rate.

49. The method of claim 48, wherein the perfusion flow rate is decreased to a minimum threshold perfusion rate required to maintain the transplant in a state evaluated to have a threshold likelihood of transplant rejection.

50. The method of claim 49, wherein the threshold likelihood is a maximum likelihood of transplant rejection.

51. The method of any of claims 47-50, wherein the perfusion parameter is a concentration of at least one component of the perfusion fluid.

52. The method of claim 51, wherein the at least one component is selected from the group of oxygen, stem cells, an immunosuppressive drug, a nutrient, or red blood cells.

53. The method of any of claims 47-52, wherein the perfusion parameter is a temperature or a pH of the perfusion fluid.

54. The method of any of claims 47-53, further comprising:
taking a post-adjustment sample of perfusion fluid; and
evaluating whether an additional adjustment is necessary.

55. The method of any of claims 1-54, wherein the donor organ is a kidney, a lung, a heart, a liver, a gallbladder, a pancreas, or an intestine.

56. The method of any of claims 1-54, wherein the donor tissue is a heart valve, a skin tissue, a bone tissue, a tendon, a cornea, a blood vessel, a cartilage tissue, a ligament, an eye tissue, or a bone marrow tissue.

57. The method of any of claims 1-54, wherein the donor tissue comprises cord blood stem cells or peripheral blood stem cells.

58. The method of any of claims 1-54, wherein the donor tissue is blood or platelets.
